(19) United States
(12) Patent Application Publication
(10) Pub. No.: US 2025/0201348 A1
(43) Pub. Date: Jun. 19, 2025

(54) ARTIFICIAL INTELLIGENCE-BASED DETECTION OF GENE CONSERVATION AND EXPRESSION PRESERVATION AT BASE RESOLUTION

(71) Applicant: ILLUMINA, INC., San Diego, CA (US)

(72) Inventor: Kishore Jaganathan, San Francisco, CA (US)

(21) Appl. No.: 18/849,927

(22) PCT Filed: Aug. 4, 2023
(86) PCT No.: PCT/US2023/029477

§ 371 Date: Sep. 23, 2024

Related U.S. Application Data
(60) Provisional application No. 63/395,778, filed on Aug. 5, 2022.

(51) Int. Cl. G16B 40/20; G16B 25/10

(52) U.S. Cl. CPC G16B 40/20; G16B 25/10

(57) ABSTRACT

The technology disclosed relates to detecting gene conservation and expression preservation. In particular, the technology disclosed relates to detecting gene conservation and epigenetic signals for a reference genetic sequence in comparison to a variant of the reference genetic sequence at base resolution through the generation of a plurality of alternative representations of the sequence in chromatin form which may represent evolutionary conservation, transcription initiation, or epigenetic signals, mapping the plurality of alternative chromatin sequences to a gene expression alterability classifier to generate a gene expression class prediction for the variant, and mapping the alternative chromatin sequence to a pathogenicity predictor to detect pathogenicity of variants.

### CROSS-REFERENCE TO RELATED APPLICATION

This application is related to U.S. Patent Application entitled “ARTIFICIAL INTELLIGENCE-BASED EPIGENETICS AT BASE RESOLUTION,” filed contemporaneously (Attorney Docket No. ILLM 1040-1/IP-2045-PRV), which is incorporated by reference for all purposes as if fully set forth herein.

### INCORPORATIONS

The following are incorporated by reference for all purposes as if fully set forth herein:
U.S. patent application Ser. No. 62/903,700, titled “ARTIFICIAL INTELLIGENCE-BASED EPIGENETICS,” filed Sep. 20, 2019 (Attorney Docket No. ILLM 1025-1/IP-1898-PRV);
Sundaram, L. et al. Predicting the clinical impact of human mutation with deep neural networks. Nat. Genet. 50, 1161-1170 (2018);
Jaganathan, K. et al. Predicting splicing from primary sequence with deep learning. Cell 176, 535-548 (2019);
U.S. Patent Application No. 62/573,144, titled “TRAINING A DEEP PATHOGENICITY CLASSIFIER USING LARGE-SCALE BENIGN TRAINING DATA,” filed Oct. 16, 2017 (Attorney Docket No. ILLM 1000-1/IP-1611-PRV);
U.S. Patent Application No. 62/573,149, titled “PATHOGENICITY CLASSIFIER BASED ON DEEP CONVOLUTIONAL NEURAL NETWORKS (CNNs),” filed Oct. 16, 2017 (Attorney Docket No. ILLM 1000-2/IP-1612-PRV);
U.S. Patent Application No. 62/573,153, titled “DEEP SEMI-SUPERVISED LEARNING THAT GENERATES LARGE-SCALE PATHOGENIC TRAINING DATA,” filed Oct. 16, 2017 (Attorney Docket No. ILLM 1000-3/IP-1613-PRV);
U.S. Patent Application No. 62/582,898, titled “PATHOGENICITY CLASSIFICATION OF GENOMIC DATA USING DEEP CONVOLUTIONAL NEURAL NETWORKS (CNNs),” filed Nov. 7, 2017 (Attorney Docket No. ILLM 1000-4/IP-1618-PRV);
U.S. patent application Ser. No. 16/160,903, titled “DEEP LEARNING-BASED TECHNIQUES FOR TRAINING DEEP CONVOLUTIONAL NEURAL NETWORKS,” filed on Oct. 15, 2018 (Attorney Docket No. ILLM 1000-5/IP-1611-US);
U.S. patent application Ser. No. 16/160,986, titled “DEEP CONVOLUTIONAL NEURAL NETWORKS FOR VARIANT CLASSIFICATION,” filed on Oct. 15, 2018 (Attorney Docket No. ILLM 1000-6/IP-1612-US);
U.S. patent application Ser. No. 16/160,968, titled “SEMI-SUPERVISED LEARNING FOR TRAINING AN ENSEMBLE OF DEEP CONVOLUTIONAL NEURAL NETWORKS,” filed on Oct. 15, 2018 (Attorney Docket No. ILLM 1000-7/IP-1613-US);
U.S. patent application Ser. No. 16/407,149, titled “DEEP LEARNING-BASED TECHNIQUES FOR PRE-TRAINING DEEP CONVOLUTIONAL NEURAL NETWORKS,” filed May 8, 2019 (Attorney Docket No. ILLM 1010-1/IP-1734-US);
U.S. patent application Ser. No. 17/232,056, titled “DEEP CONVOLUTIONAL NEURAL NETWORKS TO PREDICT VARIANT PATHOGENICITY USING THREE-DIMENSIONAL (3D) PROTEIN STRUCTURES,” filed on Apr. 15, 2021, (Atty. Docket No. ILLM 1037-2/IP-2051-US);
U.S. Patent Application No. 63/175,495, titled “MULTI-CHANNEL PROTEIN VOXELIZATION TO PREDICT VARIANT PATHOGENICITY USING DEEP CONVOLUTIONAL NEURAL NETWORKS,” filed on Apr. 15, 2021, (Atty. Docket No. ILLM 1047-1/IP-2142-PRV);
U.S. Patent Application No. 63/175,767, titled “EFFICIENT VOXELIZATION FOR DEEP LEARNING,” filed on Apr. 16, 2021, (Atty. Docket No. ILLM 1048-1/IP-2143-PRV); and
U.S. patent application Ser. No. 17/468,411, titled “ARTIFICIAL INTELLIGENCE-BASED ANALYSIS OF PROTEIN THREE-DIMENSIONAL (3D) STRUCTURES,” filed on Sep. 7, 2021, (Atty. Docket No. ILLM 1037-3/IP-2051A-US).

### FIELD OF THE TECHNOLOGY DISCLOSED

The technology disclosed relates to artificial intelligence type computers and digital data processing systems and corresponding data processing methods and products for emulation of intelligence (i.e., knowledge based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to artificial intelligence-based detection of gene conservation and expression preservation at base resolution.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling, and proteomics. Genomics arose as a data-driven science—it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions and residues such as transcriptional enhancers and single nucleotide polymorphisms (SNPs).

Genomics data are too large and too complex to be mined solely by visual investigation of pairwise correlations. For example, protein sequences can be classified into families of homologous proteins that descend from an ancestral protein and share a similar structure and function. Analyzing multiple sequence alignments (MSAs) of homologous proteins provides important information about functional and structural constraints. The statistics of MSA columns, representing amino-acid sites, identify functional residues that are conserved during evolution. Correlations of amino acid usage between the MSA columns contain important information about functional sectors and structural contacts.

Instead, analytical tools are required to support the discovery of unanticipated relationships, to derive novel hypotheses and models, and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells, or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, and the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks, and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions, are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation followed by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequences across distances as long as typical human introns (See Jaganathan, K. et al. Predicting splicing from primary sequence with deep learning. Cell 176, 535-548 (2019)).

Different types of neural networks can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model to each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to finding potential drivers of complex phenotypes. One example includes predicting the effect of non-coding single-nucleotide variants and short insertions or deletions (indels) indirectly from the difference between two variants in terms of transcription factor binding, chromatin accessibility, or gene expression predictions. Another example includes predicting novel splice site creation from sequence or quantitative effects of genetic variants on splicing.

End-to-end deep learning approaches for variant effect predictions are applied to predict the pathogenicity of missense variants from protein sequence and sequence conservation data (See Sundaram, L. et al. Predicting the clinical impact of human mutation with deep neural networks. Nat. Genet. 50, 1161-1170 (2018), referred to herein as “PrimateAI”). PrimateAI uses deep neural networks trained on variants of known pathogenicity with data augmentation using cross-species information. In particular, PrimateAI uses sequences of wild-type and mutant proteins to compare the difference and decide the pathogenicity of mutations using the trained deep neural networks. Such an approach that utilizes the protein sequences for pathogenicity prediction is promising because it can avoid the circularity problem and overfitting to previous knowledge. However, compared to the adequate number of data to train the deep neural networks effectively, the number of clinical data available in Clin Var is relatively small. To overcome this data scarcity, PrimateAI uses common human variants and variants from primates as benign data while simulated variants based on trinucleotide context were used as unlabeled data.

PrimateAI outperforms prior methods when trained directly upon sequence alignments. PrimateAI learns important protein domains, conserved amino acid positions, and sequence dependencies directly from the training data consisting of about 120,000 human samples. PrimateAI substantially exceeds the performance of other variant pathogenicity prediction tools in differentiating benign and pathogenic de-novo mutations in candidate developmental disorder genes, and in reproducing prior knowledge in ClinVar. These results suggest that PrimateAI is an important step forward for variant classification tools that may lessen the reliance of clinical reporting on prior knowledge.

Central to protein biology is the understanding of how structural elements give rise to observed function. The surfeit of protein structural data enables the development of computational methods to systematically derive rules governing structural-functional relationships. However, the performance of these methods depends critically on the choice of protein structural representation.

Protein sites are microenvironments within a protein structure, distinguished by their structural or functional role. A site can be defined by a three-dimensional (3D) location and a local neighborhood around this location in which the structure or function exists. Central to rational protein engineering is the understanding of how the structural arrangement of amino acids creates functional characteristics within protein sites. Determination of the structural and functional roles of individual amino acids within a protein provides information to help engineer and alter protein functions. Identifying functionally or structurally important amino acids allows focused engineering efforts such as site-directed mutagenesis for altering targeted protein functional properties. Alternatively, this knowledge can help avoid engineering designs that would abolish a desired function.

Since it has been established that structure is far more conserved than sequence, the increase in protein structural data provides an opportunity to systematically study the underlying pattern governing the structural-functional relationships using data-driven approaches. A fundamental aspect of any computational protein analysis is how protein structural information is represented. The performance of machine learning methods often depends more on the choice of data representation than the machine learning algorithm employed. Good representations efficiently capture the most critical information while poor representations create a noisy distribution with no underlying patterns.

The surfeit of protein structures and the recent success of deep learning algorithms provide an opportunity to develop tools for automatically extracting task-specific representations of protein structures.

The computational analysis of genomics studies is challenged by confounding variation that is unrelated to the genetic factors of interest. Identification of variants that cause extreme levels of gene expression, either high or low, is paramount to the diagnosis of the pathogenicity of genetic diseases. However, there are numerous confounding factors that can interfere with the identification of pathogenic variants. Isolating variants by examining rare variants that can be associated with specific pathologies can simplify the problem. Further, removing noise introduced by confounders can increase the signal-to-noise ratio.

Therefore, an opportunity arises to apply artificial intelligence to epigenetics to greatly increase the sensitivity in recovering genetic associations between variable genetic loci and the expression levels of individual genes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

In the drawings, like reference characters generally refer to like parts throughout the different views. Also, the drawings are not necessarily to scale, with an emphasis instead generally being placed upon illustrating the principles of the technology disclosed. In the following description, various implementations of the technology disclosed are described with reference to the following drawings, in which.

FIG. 1 is a flow diagram that illustrates a process of a system for determining evolutionary and epigenetic characteristics of a genetic sequence.

FIG. 2 schematically illustrates an example input base sequence comprising nucleotide bases extracted from a sequence database, in which a target base sequence is flanked by a left sequence containing upstream context bases, and a right sequence containing downstream context bases.

FIG. 3 illustrates an example of alternate sequences from an example reference genetic sequence with two example alternate sequences and in which the alternate sequences possess a respective single nucleotide variant in a single base position but otherwise possess an identical composition to the reference sequence.

FIG. 4 illustrates the genetic composition of sequences belonging to training datasets developed for one implementation of the technology disclosed.

FIG. 5 schematically illustrates one implementation of a training procedure applied to the system of FIG. 1 with the model being trained with the first training set described in FIG. 4 and then undergoing re-training with the second training set described in FIG. 4.

FIG. 6 schematically illustrates another implementation of a training procedure applied to the system of FIG. 1 with the first training set described in FIG. 4 and then undergoing re-training with a subset of the second training set described in FIG. 4, followed by model validation with the remaining subset of samples from the second training set.

FIG. 7 is a schematic diagram of an implementation of the system from FIG. 1 for variant classification wherein the system is used to compare a reference sequence and alternate sequence at base resolution by comparing the respective model outputs for each sequence.

FIG. 8 is a flow diagram of one implementation of the technology disclosed in which the comparison of the reference sequence and the alternate sequence is quantified by an average delta value.

FIG. 9 is a flow diagram of one implementation of the technology disclosed in which the comparison of the reference sequence and the alternate sequence is quantified by a final sum delta value.

FIG. 10 is a flow diagram of one implementation of the technology disclosed in which the biological quantities model generates two biological quantities output sequences from an input base sequence via a first set of weights and a second set of weights which are trained end-to-end.

FIG. 11 is a flow diagram of one implementation of the technology disclosed in which the biological quantities model generates three biological quantities output sequences from an input base sequence via a first set of weights and a second set of weights which are trained end-to-end.

FIG. 12 is a flow diagram of one implementation of the technology disclosed in which the biological quantities model comprises a first set of weights that generates an alternate representation of the input base sequence and a second set of weights that is trained from scratch to generate a plurality of biological quantities output sequences from the alternate representation of the input base sequence.

FIG. 13 is a flow diagram of one implementation of the technology disclosed in which the biological quantities model generates one biological quantities output sequence from an input base sequence via a first set of weights and a second set of weights which are trained end-to-end and retrained to generate succeeding biological quantities output sequences on a singular basis.

FIG. 14 is a flow diagram of one implementation of the technology disclosed in which the biological quantities model comprises a first set of weights and second set of weights which are trained end-to-end to generate a plurality of biological quantities output sequences from an input base sequence, and a third and fourth set of weights which are trained end-to-end to generate a gene expression output sequence from the input plurality of biological quantities output sequences.

FIG. 15 is a flow diagram of one implementation of the technology disclosed in which the biological quantities model comprises a first set of weights that generates an alternative representation of the input base sequence, a second set of weights that is trained from scratch to generate a plurality of biological quantities output sequences from the alternative representation of the input base sequence, and a third and fourth set of weights which are trained end-to-end to generate a gene expression output sequence from the input plurality of biological quantities output sequences.

FIG. 16 is a flow diagram of one implementation of the technology disclosed in which the biological quantities model comprises a first set of weights that generates an alternate representation of the input base sequence, a second set of weights that is trained from scratch to generate a plurality of biological quantities output sequences, a third set of weights that is trained from scratch to generate an alternative biological quantities representation from the plurality of biological quantities output sequences, and a fourth set of weights that is trained from scratch to generate a gene expression output sequence from the alternative biological quantities representation.

FIG. 17 is a flow diagram of one implementation of the technology disclosed in which the biological quantities model comprises a first set of weights and second set of weights which are trained end-to-end to generate a plurality of biological quantities output sequences from an input base sequence, a third set of weights that is trained from scratch to generate an alternative biological quantities representation from the plurality of biological quantities output sequences, and a fourth set of weights that is trained from scratch to generate a gene expression output sequence from the alternative biological quantities representation.

FIG. 18 is a flow diagram of one implementation of the technology disclosed in which the biological quantities model comprises a first set of weights that is trained to generate a plurality of biological quantities output sequences from an input base sequence, and then retrained as a substitute of the third set of weights to generate an alternative biological quantities representation from the plurality of biological quantities output sequences, and a fourth set of weights that is trained end-to-end with the substituted first set of weights to generate a gene expression output sequence from the alternative biological quantities representation.

FIG. 19 is a flow diagram of one implementation of the technology disclosed in which the biological quantities model comprises a first set of weights that is trained to generate an alternative sequence representation from an input base sequence, and then retrained as a substitute of the third set of weights to generate an alternative biological quantities representation from the plurality of biological quantities output sequences, and a fourth set of weights that is trained from scratch to generate a gene expression output sequence from the alternative biological quantities representation.

FIG. 20 is a flow diagram of one implementation of the technology disclosed in which the biological quantities model comprises a first set of weights that is trained to generate an alternative sequence representation from an input base sequence, a second set of weights that is trained to generate a plurality of biological quantities output sequences from the alternate representation of the input base, the retrained first set of weights used as a substitute of the third set of weights to generate an alternative biological quantities representation from the plurality of biological quantities output sequences, and a fourth set of weights that is trained from scratch to generate a gene expression output sequence from the alternative biological quantities representation.

FIG. 21 is a flow diagram of one implementation of the technology disclosed in which the biological quantities model comprises a first set of weights that is trained to generate an alternative sequence representation from an input base sequence, a second set of weights that is trained to generate a plurality of biological quantities output sequences from the alternate representation of the input base, the retrained first set of weights used as a substitute of the third set of weights to generate an alternative biological quantities representation from the plurality of biological quantities output sequences, and a fourth set of weights that is trained end-to-end with the substituted first set of weights to generate a gene expression output sequence from the alternative biological quantities representation.

FIG. 22 is a flow diagram of one implementation of the technology disclosed in which the model is further configured to comprise a pathogenicity prediction logic that compares the reference biological quantities output sequence and the alternate biological quantities output sequence at base resolution wherein a fifth set of weights generates an alternate sequence pathogenicity prediction from the plurality of biological quantities output sequences wherein the first and second set of weights are each trained from scratch.

FIG. 23 is a flow diagram of one implementation of the technology disclosed in which the model is further configured to comprise a pathogenicity prediction logic that compares the reference biological quantities output sequence and the alternate biological quantities output sequence at base resolution wherein a fifth set of weights generates an alternate sequence pathogenicity prediction from the plurality of biological quantities output sequences wherein the first and second set of weights are trained end-to-end.

FIG. 24 is a schematic of the measures of evolutionary conservation that can be generated from the biological quantities model from the input base sequence as a value for the first biological quantities output sequence.

FIG. 25 is a schematic of the measure of transcription initiation that can be generated from the biological quantities model from the input base sequence as a value for the second biological quantities output sequence.

FIG. 26 is a schematic of the epigenetic signals that can be generated from the biological quantities model from the input base sequence as a value for the third biological quantities output sequence.

FIG. 27 is a flow diagram of one implementation of the technology disclosed in which an expression alteration classifier is configured to predict the effect of a variant on gene expression.

FIG. 28 is a flow diagram of one implementation of the technology disclosed in which the expression alteration classifier is further configured as an expression reduction classifier to predict if a variant reduces gene expression or does not reduce gene expression.

FIG. 29 is a flow diagram of one implementation of the technology disclosed in which the expression alteration classifier is further configured as an expression reduction classifier to predict if a variant increases gene expression or does not increase gene expression.

FIG. 30 is a flow diagram of one implementation of the technology disclosed in which the expression alteration classifier is further configured into a multi-class expression classifier that predicts if a variant preserves gene expression, reduces gene expression, or increases gene expression.

FIG. 31 is a flow diagram of one implementation of the technology disclosed in which gene expression classifier training is employed for the comparison of the ground truth causality scores to the inferred causality scores.

FIG. 32 shows an example computer system that can be used to implement the technology disclosed.

### DETAILED DESCRIPTION

The following discussion is presented to enable any person skilled in the art to make and use the technology disclosed and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown but is to be accorded the widest scope consistent with the principles and features disclosed herein.

The detailed description of various implementations will be better understood when read in conjunction with the appended drawings. To the extent that the figures illustrate diagrams of the functional blocks of the various implementations, the functional blocks are not necessarily indicative of the division between hardware circuitry. Thus, for example, one or more of the functional blocks (e.g., modules, processors, or memories) may be implemented in a single piece of hardware (e.g., a general purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various implementations are not limited to the arrangements and instrumentality shown in the drawings.

The processing engines and databases of the figures, designated as modules, can be implemented in hardware or software, and need not be divided up in precisely the same blocks as shown in the figures. Some of the modules can also be implemented on different processors, computers, or servers, or spread among a number of different processors, computers, or servers. In addition, it will be appreciated that some of the modules can be combined, operated in parallel or in a different sequence than that shown in the figures without affecting the functions achieved. The modules in the figures can also be thought of as flowchart steps in a method. A module also need not necessarily have all its code disposed contiguously in memory; some parts of the code can be separated from other parts of the code with code from other modules or other functions disposed in between.

### Example Applications of the Technology Disclosed

As emphasized by the numerous comparisons of various implementations of the biological quantities model 124, many implementations share an overlap in architectural components. Regarding the phrase “biological quantities model,” a biological quantities model predicts a plurality of classes of biological quantities from a genomic sequence. Examples of the classes of biological quantities include protein (transcription factor) binding, methylation, histone modifications, DNA accessibility, conservation. Of these, methylation and histone modifications count as epigenetics. In contrast, chromatin refers to histone modifications and DNA accessibility.

Accordingly, in some implementations, a biological quantities model can be referred to as an “epigenetics model.” In other implementations, a biological quantities model can be referred to as a “chromatin model.” In yet other implementations, a biological quantities model can be referred to as a “chromatin and epigenetics model” or “epigenetics and chromatin model.”

Each element of the biological quantities model 124 has multiple implementations which can be combined in numerous configurations. The multiple permutations which can be implemented for the technology disclosed provides both a broader range of utility, performance efficiency, and performance accuracy. The data transformation applied to the input base sequence in many implementations of the technology disclosed to generate of a plurality of additional sequence formats from the perspective of nucleic acid sequence and the perspective of chromatin structure is an innovative strategy that results in the output of a surfeit of output signals with broad applicability to a wide range of genomics, protein analysis, and pathogenicity research questions. Previous versions of PrimateAI have employed multiple tools for the classification of variant pathogenicity with high performance. This biological quantities model 124 introduces another tool in this methodology as well as an additional dimension with the study of epigenetic signals affecting biological replication and transcription processes.

Although there is clear utility for the additional vantage point of chromatin structure to study gene expression to the variety of tools provided by PrimateAI, the true impact provided by the technology disclosed lies in the addition of epigenetic signals to the overall gene expression prediction logic. Both the DNA sequence and histone protein components of chromatin can undergo a plethora of chemical modifications. Enzymes that bind directly to chromatin components and catalyze chemical modifications of the chromatin components can alter chromatin structure, and changes in chromatin structure can also alter the ability of chromatin-interacting enzymes to access their target ligands and function. The structure of chromatin and the enzymes that alter structure directly influence the accessibility of a gene for transcription and expression. DNA variants can cause changes in chromatin structure which subsequently may change epigenetic effects such as transcription factor binding and enzymatic reactions necessary for the proper regulation of gene expression and gene suppression.

Inversely, epigenetic effects on chromatin such as methylation and protein binding events can affect mutation rate, potentially introducing variants that may be silent or pathogenic. The study of evolutionary constraint on a gene and pathogenicity of variants of that gene is significantly more comprehensive and accurate when augmented by epigenetic features as demonstrated in many implementations of the technology disclosed. Overall, the technology disclosed possesses several permutations which are amenable to a range of training and learning strategies to generate several outputs which can be applied to the prediction of gene expression and gene pathogenicity for a target genetic sequence. The chromatin-focused strategy disclosed is useful in the study of inherited and environmental exposure-related disease, the development of drugs, and the influence of epigenetics in the transcription and translation of nucleic acid sequences to proteins.

### Biological Quantities model Overview

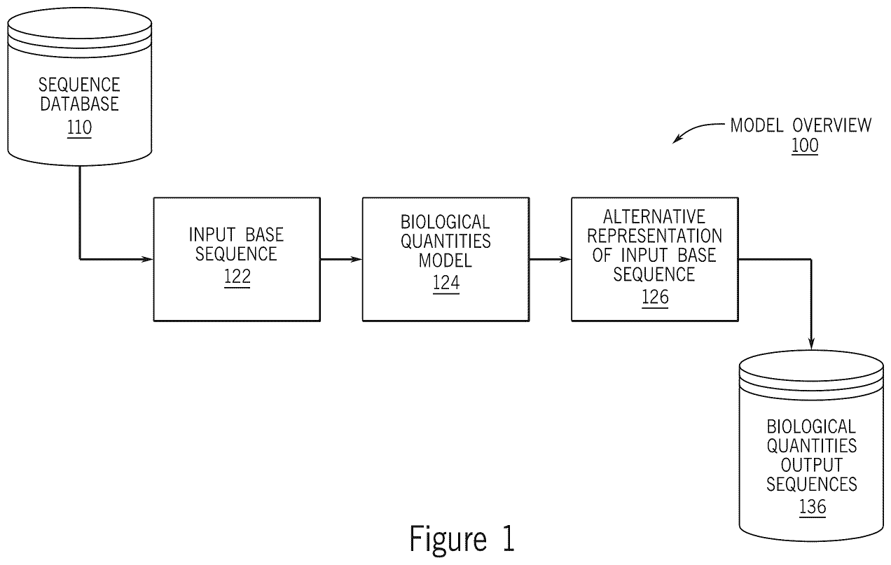

FIG. 1 is a flow diagram that illustrates a process 100 of a system for determining evolutionary and epigenetic characteristics of a genetic sequence. An input base sequence 122 is extracted from a sequence database 110 and processed by a biological quantities model 124 that generates an alternative representation of the input base sequence 126. The alternative representation of the input base sequence 126 is converted into an alternative biological quantities representation in the form of a plurality of biological quantities output sequences 136.

### Biological Quantities Model Input Data

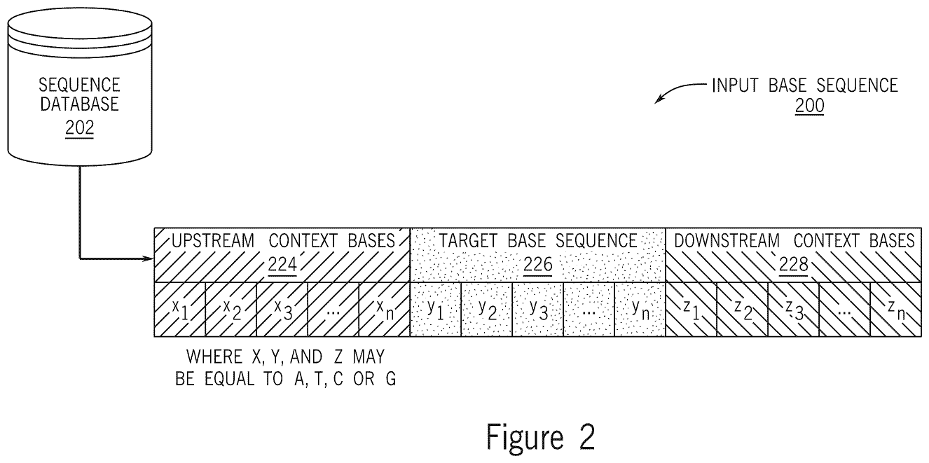

FIG. 2 schematically illustrates an example input base sequence 200 comprising nucleotide bases extracted from a sequence database 202, in which a target base sequence 226 is flanked by a left sequence 224 containing upstream context bases, and a right sequence 228 containing downstream context bases. The upstream context bases 224 are a sequence of nucleotide bases {x₁, x₂, x₃, . . . , xₙ} which can be equal to adenine, thymine, cytosine, or guanine. The target base sequence 226 subsequently follows the upstream context bases 224. The target base sequence 226 contains a sequence of nucleotide bases {y₁, y₂, y₃, . . . , yₙ} which can be which can be equal to adenine, thymine, cytosine, or guanine. The downstream context bases 228 subsequently follow the target base sequence 226. The downstream context bases are a sequence of nucleotide bases {z₁, z₂, z₃, . . . , zₙ} which can be which can be equal to adenine, thymine, cytosine, or guanine. An input base sequence 200 may also contain an unknown or missing gap position in the upstream context bases 224, target base sequence 226, or downstream context bases 228.

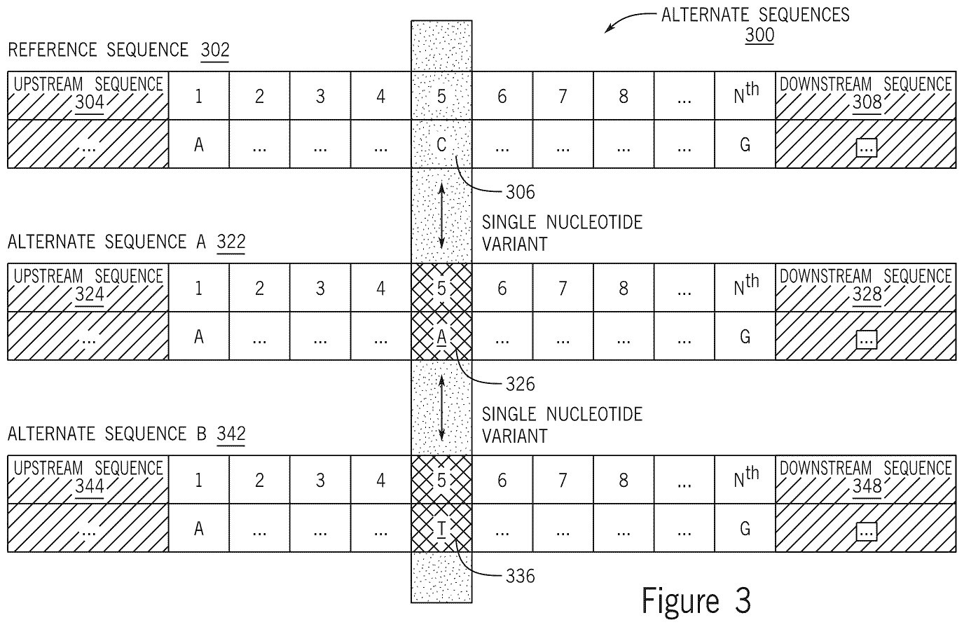

FIG. 3 illustrates an example of alternate sequences 300 from an example reference genetic sequence 302 with two example alternate sequences 322 and 342 in which the alternate sequences possess a respective single nucleotide variant in a single base position but otherwise possess an identical composition to the reference sequence. For example, a single nucleotide substitution is shown as variant 326 and variant 336 as compared to nucleotide 306. In addition to possessing identical target base sequences, upstream sequences 304, 324, and 344 are identical to each other and downstream sequences 308, 328, and 348 are identical to each other.

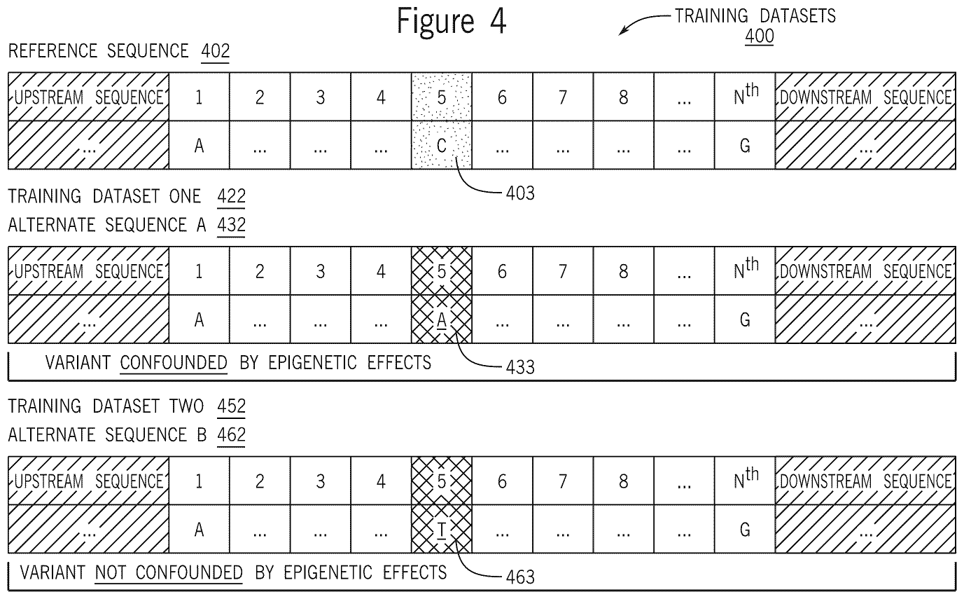

FIG. 4 illustrates the genetic composition of sequences belonging to training datasets 400 developed for one implementation of the technology disclosed. One training dataset 422 contains alternate sequences (e.g., alternate sequence A 432 possessing a single nucleotide variant 433) which are confounded by epigenetic effects, and a second training dataset 452 contains alternate sequences (e.g., sequence 462 possessing a single nucleotide variant 463) which are not confounded by epigenetic effects. The single nucleotide variants 433 and 463 differ in composition from the reference base position 403; however, all other base positions within reference sequence, alternate sequence A 432 and alternate sequence B 462 do not differ.

### Biological Quantities Model Structure

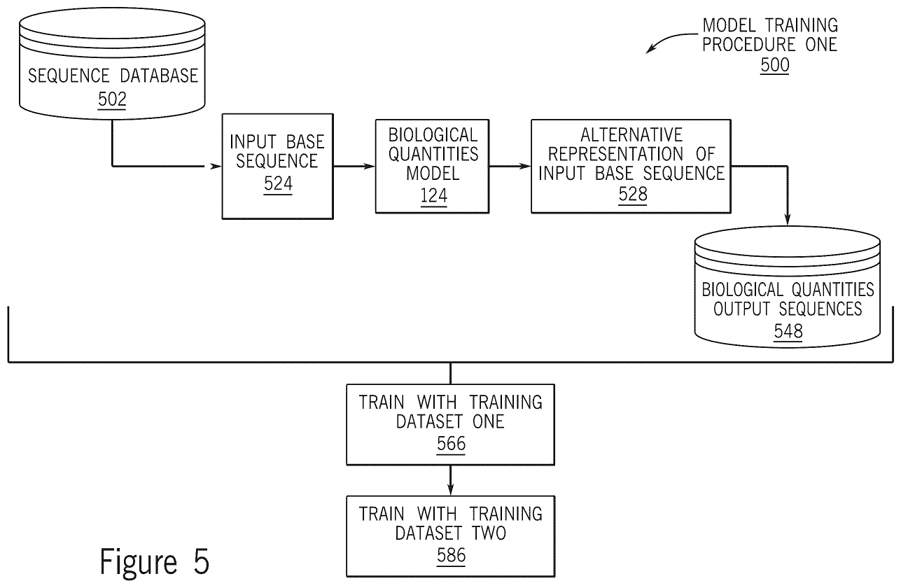

FIG. 5 schematically illustrates one implementation 500 of a training procedure applied to the system 100 of FIG. 1 with the biological quantities model 124 being trained with the first training set described in FIG. 4 and then undergoing re-training with the second training set described in FIG. 4.

The sequences obtained from sequence database 502 are first obtained from the first training dataset for the first set of training iterations 566 to generate a plurality biological quantities output sequences 548 from an input base sequence 524 with the biological quantities model 124 configured to detect changes in gene expression at base resolution, comprising the biological quantities model 124 that processes the input base sequence 524 and generates an alternative representation (e.g., a convolved representation) of the input base sequence 524, and a biological quantities output sequence generator 528. The biological quantities model 124 then undergoes a second set of training iterations 586 on sequences obtained from the second training dataset without any changes to the model configuration of the biological quantities model 124.

In one implementation, the biological quantities model 124 contains groups of residual blocks arranged in a sequence from lowest to highest. Each group of residual blocks is parameterized by a number of convolution filters in the residual blocks, a convolution window size of the residual blocks, and an atrous convolution rate of the residual blocks. The atrous convolution rate progresses non-exponentially from a lower residual block group to a higher residual block group, in some implementations. In other implementations, it progresses exponentially. The size of convolution window varies between groups of residual blocks, and each residual block comprises at least one batch normalization layer, at least one rectified linear unit (abbreviated ReLU) layer, at least one atrous convolution layer, and at least one residual connection.

In one implementation, the dimensionality of the input is (Cu+L+Cd)×4, where Cu is a number of upstream flanking context bases, Cd is a number of downstream flanking context bases, and L is a number of bases in the input promoter sequence. The dimensionality of the output is 4×L. In some implementations, each group of residual blocks produces an intermediate output by processing a preceding input and the dimensionality of the intermediate output is (I−[{(W−1)*D}*A])×N, where I is dimensionality of the preceding input, W is convolution window size of the residual blocks, D is atrous convolution rate of the residual blocks, A is a number of atrous convolution layers in the group, and N is a number of convolution filters in the residual blocks.

In one implementation, the input has 200 upstream flanking context bases (Cu) to the left of the input sequence and 200 downstream flanking context bases (Cd) to the right of the input sequence. The length of the input sequence (L) can be arbitrary, such as 3001. In one implementation, each residual block in a first group has 32 convolution filters, 11 convolution window size, and 1 atrous convolution rate and each residual block in a second group has 32 convolution filters, 11 convolution window size, and 4 atrous convolution rate. In other architectures, each residual block has 32 convolution filters, 11 convolution window size, and 1 atrous convolution rate.

In one implementation, the input has one thousand upstream flanking context bases (Cu) to the left of the input sequence and one thousand downstream flanking context bases (Cd) to the right of the input sequence. The length of the input sequence (L) can be arbitrary, such as 3001. In one implementation, there are at least three groups of four residual blocks and at least three skip connections. Each residual block in a first group has 32 convolution filters, 11 convolution window size, and 1 atrous convolution rate, each residual block in a second group has 32 convolution filters, 11 convolution window size, and 4 atrous convolution rate, and each residual block in a third group has 32 convolution filters, 21 convolution window size, and 19 atrous convolution rate.

In one implementation, the input has five thousand upstream flanking context bases (Cu) to the left of the input sequence and five thousand downstream flanking context bases (Cd) to the right of the input sequence. The length of the input sequence (L) can be arbitrary, such as 3001. In one implementation, there are at least four groups of four residual blocks and at least four skip connections. Each residual block in a first group has 32 convolution filters, 11 convolution window size, and 1 atrous convolution rate, each residual block in a second group has 32 convolution filters, 11 convolution window size, and 4 atrous convolution rate, each residual block in a third group has 32 convolution filters, 21 convolution window size, and 19 atrous convolution rate, and each residual block in a fourth group has 32 convolution filters, 41 convolution window size, and 25 atrous convolution rate.

Generally speaking, the biological quantities model 124 can be a rule-based model, a tree-based model, or a machine learning model. Examples include a multilayer perceptron (MLP), a feedforward neural network, a fully-connected neural network, a fully convolution neural network, a ResNet, a sequence-to-sequence (Seq2Seq) model like WaveNet, a semantic segmentation neural network, and a generative adversarial network (GAN) (e.g., CycleGAN, StyleGAN, pixelRNN, text-2-image, DiscoGAN, IsGAN).

In some implementations, the biological quantities model 124 can include self-attention mechanisms like Transformer, Vision Transformer (ViT), Bidirectional Transformer (BERT), Detection Transformer (DETR), Deformable DETR, UP-DETR, DeiT, Swin, GPT, iGPT, GPT-2, GPT-3, BERT, SpanBERT, ROBERTa, XLNet, ELECTRA, UniLM, BART, T5, ERNIE (THU), KnowBERT, DeiT-Ti, DeiT-S, DeiT-B, T2T-ViT-14, T2T-ViT-19, T2T-ViT-24, PVT-Small, PVT-Medium, PVT-Large, TNT-S, TNT-B, CPVT-S, CPVT-S-GAP, CPVT-B, Swin-T, Swin-S, Swin-B, Twins-SVT-S, Twins-SVT-B, Twins-SVT-L, Shuffle-T, Shuffle-S, Shuffle-B, XCIT-S12/16, CMT-S, CMT-B, VOLO-D1, VOLO-D2, VOLO-D3, VOLO-D4, MoCo v3, ACT, TSP, Max-DeepLab, VisTR, SETR, Hand-Transformer, HOT-Net, METRO, Image Transformer, Taming transformer, TransGAN, IPT, TTSR, STTN, Masked Transformer, CLIP, DALL-E, Cogview, UniT, ASH, TinyBert, FullyQT, ConvBert, FCOS, Faster R-CNN+FPN, DETR-DC5, TSP-FCOS, TSP-RCNN, ACT+MKDD (L=32), ACT+MKDD (L=16), SMCA, Efficient DETR, UP-DETR, UP-DETR, ViTB/16-FRCNN, ViT-B/16-FRCNN, PVT-Small+RetinaNet, Swin-T+RetinaNet, Swin-T+ATSS, PVT-Small+DETR, TNT-S+DETR, YOLOS-Ti, YOLOS-S, and YOLOS-B.

In some implementations, examples of the biological quantities model 124 include a convolution neural network (CNN) with a plurality of convolution layers, a recurrent neural network (RNN) such as a long short-term memory network (LSTM), bi-directional LSTM (Bi-LSTM), or a gated recurrent unit, and a combination of both a CNN and an RNN.

In some implementations, the biological quantities model 124 can use ID convolutions, 2D convolutions, 3D convolutions, 4D convolutions, 5D convolutions, dilated or atrous convolutions, transpose convolutions, depthwise separable convolutions, pointwise convolutions, 1×1 convolutions, group convolutions, flattened convolutions, spatial and cross-channel convolutions, shuffled grouped convolutions, spatial separable convolutions, and deconvolutions. The biological quantities model 124 can use one or more loss functions such as logistic regression/log loss, multi-class cross-entropy/softmax loss, binary cross-entropy loss, mean-squared error loss, L1 loss, L2 loss, smooth L1 loss, and Huber loss. The biological quantities model 124 can use any parallelism, efficiency, and compression schemes such TFRecords, compressed encoding (e.g., PNG), sharding, parallel calls for map transformation, batching, prefetching, model parallelism, data parallelism, and synchronous/asynchronous stochastic gradient descent (SGD). The biological quantities model 124 can include upsampling layers, downsampling layers, recurrent connections, gates and gated memory units (like an LSTM or GRU), residual blocks, residual connections, highway connections, skip connections, peephole connections, activation functions (e.g., non-linear transformation functions like rectifying linear unit (ReLU), leaky ReLU, exponential liner unit (ELU), sigmoid and hyperbolic tangent (tanh)), batch normalization layers, regularization layers, dropout, pooling layers (e.g., max or average pooling), global average pooling layers, and attention mechanisms.

In some implementations, the biological quantities model 124 can be a linear regression model, a logistic regression model, an Elastic Net model, a support vector machine (SVM), a random forest (RF), a decision tree, or a boosted decision tree (e.g., XGBoost), or some other tree-based logic (e.g., metric tree, kd-tree, R-tree, universal B-tree, X-tree, ball tree, locality sensitive hash, and inverted index). The biological quantities model 124 can be an ensemble of multiple models, in some implementations.

In some implementations, the biological quantities model 124 can be trained using backpropagation-based gradient update techniques. Example gradient descent techniques that can be used for training the models include stochastic gradient descent, batch gradient descent, and mini-batch gradient descent. Some examples of gradient descent optimization algorithms that can be used to train the models are Momentum, Nesterov accelerated gradient, Adagrad, Adadelta, RMSprop, Adam, AdaMax, Nadam, and AMSGrad.

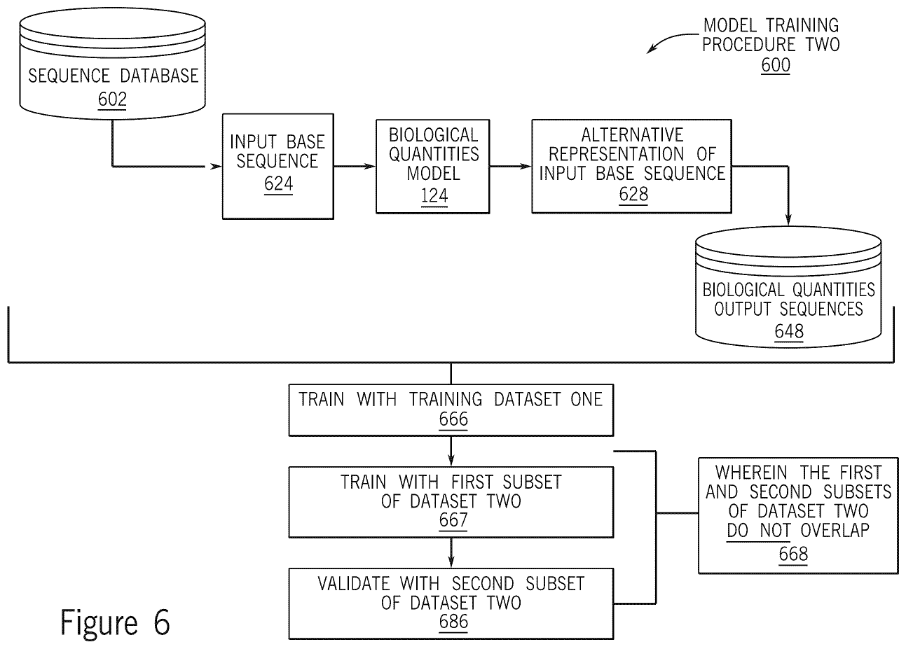

FIG. 6 schematically illustrates another implementation 600 of a training procedure applied to the system 100 of FIG. 1 with the first training set described in FIG. 4 and then undergoing re-training with a subset of the second training set described in FIG. 4, followed by model validation of the biological quantities model 124 with the remaining subset of samples from the second training set. The sequences obtained from sequence database 602 are first obtained from the first training dataset for the first set of training iterations 666 to generate a plurality biological quantities output sequences 648 from an input base sequence 624 with the biological quantities model 124 configured to detect changes in gene expression at base resolution, comprising the biological quantities model 124 that processes the input base sequence 624 and generates an alternative representation (e.g., a convolved representation) of the input base sequence 624, and a biological quantities output sequence generator 628. The biological quantities model 124 then undergoes a second set of training iterations 667 on a subset of sequences obtained from the second training dataset without any changes to the model configuration of the biological quantities model 124. Following training iterations 666 and training iterations 667, the biological quantities model 124 undergoes a validation process 686 with a second subset of the second training dataset wherein the first and second subsets of training dataset two do not overlap 668, which allows the biological quantities model 124 to be trained on more diverse and unseen training examples.

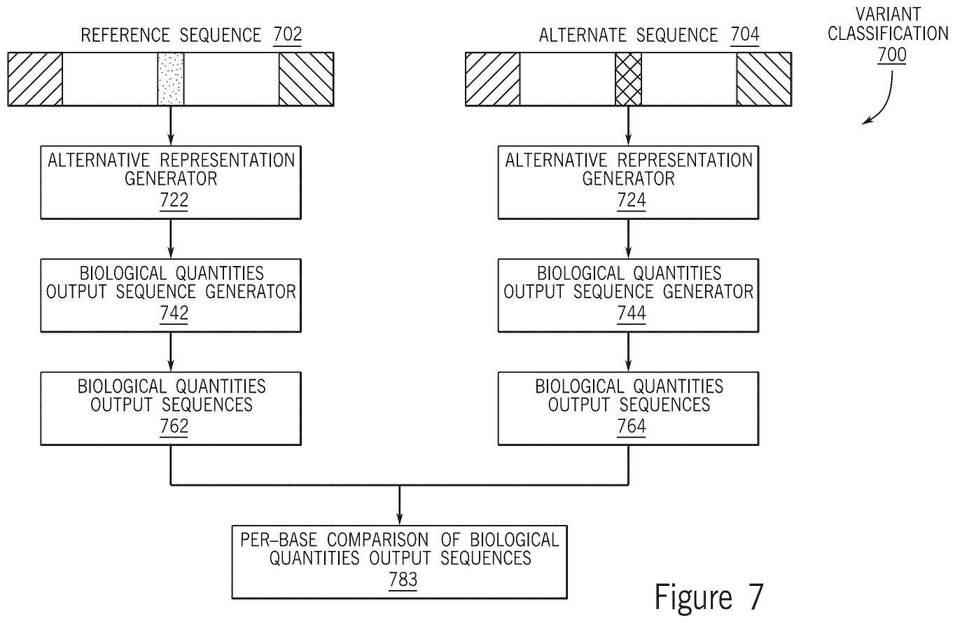

FIG. 7 is a schematic diagram of an implementation 700 of the system 100 from FIG. 1 for variant classification wherein the system is used to compare a reference sequence 702 and alternate sequence 704 at base resolution by comparing the respective model outputs of the biological quantities model 124 for each sequence represented in 783. The reference sequence 702 is separately processed by the biological quantities model 124. An alternative representation generator 722 processes the reference input base sequence 702 to generate an alternative representation (e.g., a convolved representation sequence), and a biological quantities output sequence generator 742 processes the alternative representation to generate a plurality of biological quantities output sequences 762. The alternate sequence 704 is separately processed by the biological quantities model 124. An alternative representation generator 724 processes the reference input base sequence 704 to generate an alternative representation (e.g., a convolved representation sequence), and a biological quantities output sequence generator 744 processes the alternative representation to generate a plurality of biological quantities output sequences 764. As demonstrated in the process represented terminating the flow diagram 783, the plurality of biological quantities output sequences 762 and plurality of biological quantities output sequences 764 are compared at base resolution.

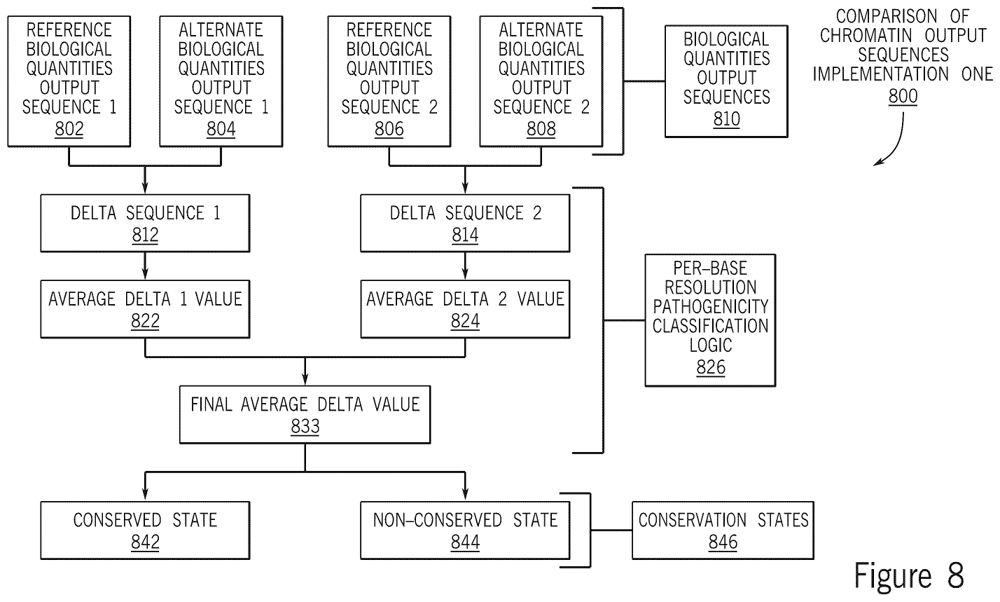

FIG. 8 is a flow diagram of one implementation 800 of the technology disclosed in which the comparison of the reference sequence and the alternate sequence is quantified by a final average delta value 833. To determine the final average delta value 833, a base resolution pathogenicity classification logic 826 is configured to process differences between a plurality of biological quantities output sequences 810 predicted for the reference sequence and the alternate sequence. The final average delta value 833 is taken as an average from a first accumulated average delta value 822 comparing the reference sequence and alternate sequence and a second average delta value 824 comparing the reference sequence and alternate sequence. A first delta sequence 812 is generated as the per-base difference between a first reference biological quantities output sequence 802 predicted from the reference sequence and a first alternate biological quantities output sequence 804 predicted from the alternate sequence. A second delta sequence 814 is generated as the per-base difference between a second reference biological quantities output sequence 806 and a second alternate biological quantities output sequence 808. The first accumulated average delta value 822 is taken as the average of the delta values obtained for each base position within the first delta sequence 812. The second accumulated average delta value 824 is taken as the average of the delta values obtained for each base position within the second delta sequence 814. Based on the final average delta value 833, a variant represented by the alternate sequence can be classified into conservation states 846, wherein a variant may be classified as belonging to a conserved state 842 or a non-conserved state 844.

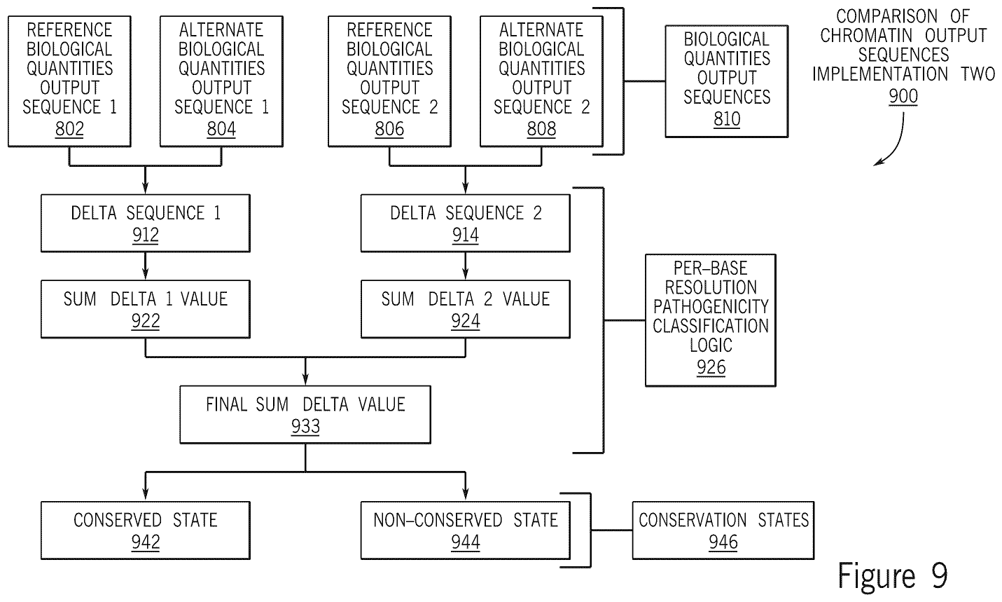

FIG. 9 is a flow diagram of one implementation 900 of the technology disclosed in which the comparison of the reference sequence and the alternate sequence is quantified by a final sum delta value 933. To determine the final sum delta value 933, a base resolution pathogenicity classification logic 926 is configured to process differences between a plurality of biological quantities output sequences 910 predicted for the reference sequence and the alternate sequence. The final sum delta value 933 is taken as a sum from a first accumulated sum delta value 922 comparing the reference sequence and alternate sequence and a second sum delta value 924 comparing the reference sequence and alternate sequence. A first delta sequence 912 is generated as the per-base difference between a first reference biological quantities output sequence 902 predicted from the reference sequence and a first alternate biological quantities output sequence 904 predicted from the alternate sequence. A second delta sequence 914 is generated as the per-base difference between a second reference biological quantities output sequence 906 and a second alternate biological quantities output sequence 909. The first accumulated sum delta value 922 is taken as the sum of the delta values obtained for each base position within the first delta sequence 912. The second accumulated sum delta value 924 is taken as the sum of the delta values obtained for each base position within the second delta sequence 914. Based on the final sum delta value 933, a variant represented by the alternate sequence can be classified into conservation states 946, wherein a variant may be classified as belonging to a conserved state 942 or a non-conserved state 944.

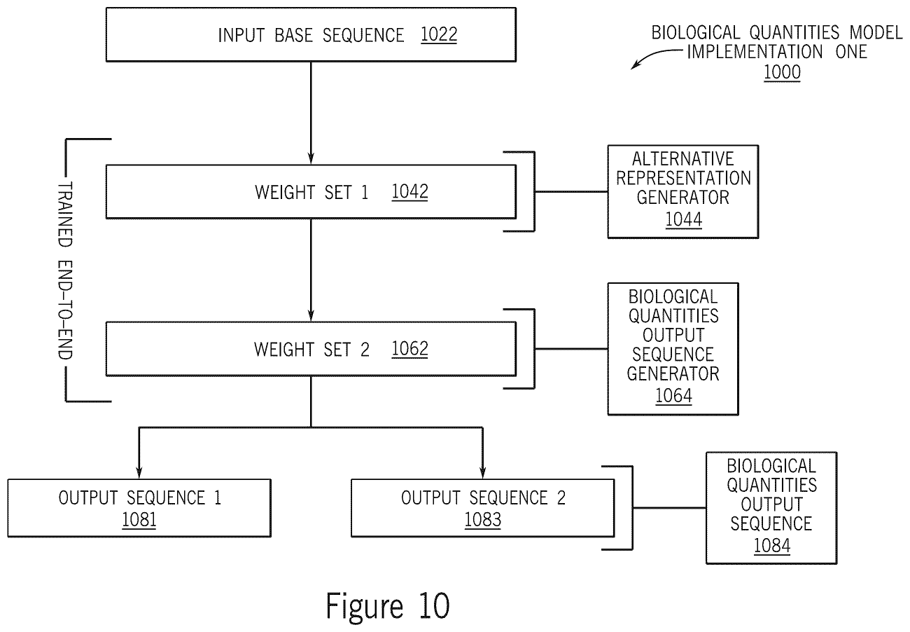

FIG. 10 is a flow diagram of one implementation 1000 of the technology disclosed in which the biological quantities model 124 generates two biological quantities output sequences 1084 from an input base sequence 1022 via a first set of weights 1042 and a second set of weights 1062 which are trained end-to-end. The first set of weights 1042 comprise an alternative representation generator 1044 that processes the input base sequence 1022 to generate an alternative representation of the input base sequence 1022. The second set of weights 1062 comprise a biological quantities output sequence generator 1064 that processes the alternative representation of the input base sequence 1022 and generates a plurality of biological quantities output sequences 1084. In implementation 1000 of the technology disclosed, two output sequences 1081 and 1083 are generated from the biological quantities output sequence generator 1064.

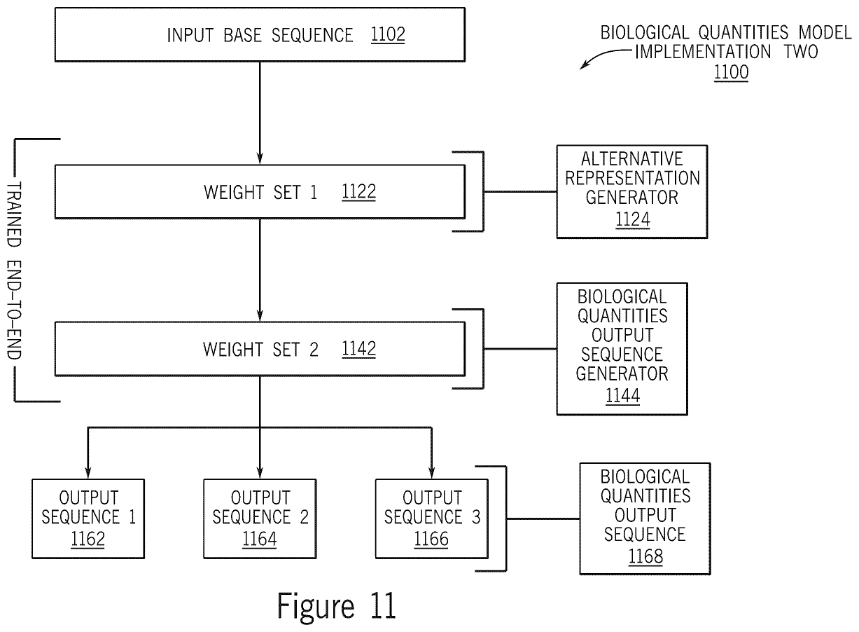

FIG. 11 is a flow diagram of one implementation 1100 of the technology disclosed in which the biological quantities model 124 generates three biological quantities output sequences 1168 from an input base sequence 1102 via a first set of weights 1122 and a second set of weights 1142 which are trained end-to-end. The first set of weights 1122 comprise an alternative representation generator 1124 that processes the input base sequence 1102 to generate an alternative representation of the input base sequence 1102. The second set of weights 1142 comprise a biological quantities output sequence generator 1144 that processes the alternative representation of the input base sequence 1102 and generates a plurality of biological quantities output sequences 1168.

Implementation 1100 in FIG. 11 differs from implementation 1000 in FIG. 10 in that three output sequences 1162, 1164, and 1166 are generated from the biological quantities output sequence generator 1144 in comparison to the two output sequences 1081 and 1083 generated from the biological quantities output sequence generator 1064. In FIG. 10, the first output sequence 1081 may be a per base measure of evolutionary conservation. The second output sequence 1083 may be a per base measure of transcription initiation. In FIG. 11, the first output sequence 1162 may be a per base measure of evolutionary conservation. The second output measure 1164 may be a per base measure of transcription initiation.

A person skilled in the art will appreciate that multiple output sequences can be predicted simultaneously and can representing different permutations and combinations of information, such as the measure of evolutionary conservation, the measure of transcription initiation represented, and the per base epigenetic signal.

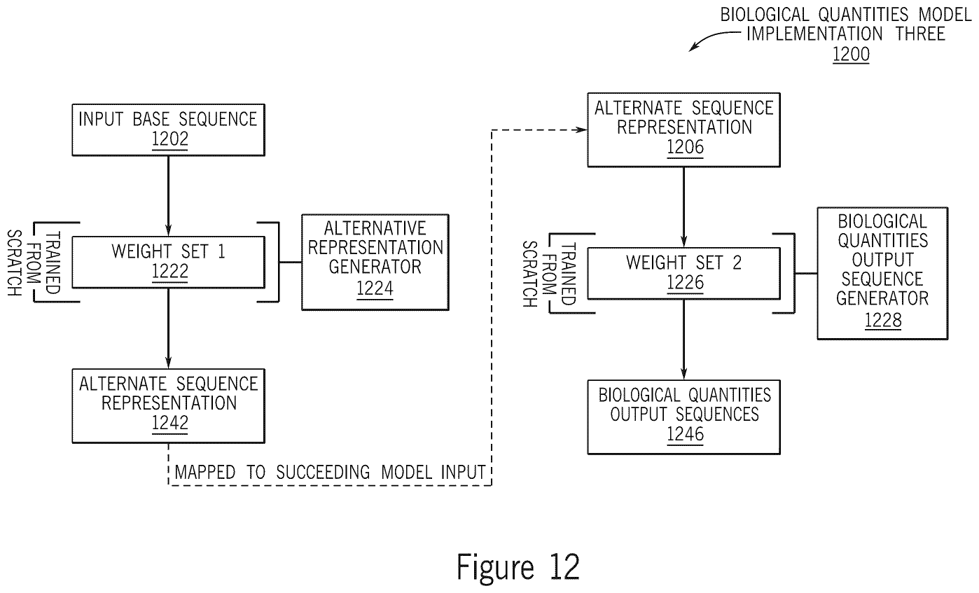

FIG. 12 is a flow diagram of one implementation 1200 of the technology disclosed in which the biological quantities model 124 comprises a first set of weights 1222 that generates an alternate representation of the input base sequence 1242 and a second set of weights 1226 that is trained from scratch to generate a plurality of biological quantities output sequences 1246 from the alternate representation of the input base sequence 1206. In comparison to implementation 1000 in FIG. 10 and implementation 1100 in FIG. 11, the first set of weights and second set of weights are not trained end-to-end in implementation 1200. As done similarly in implementation 1000 in FIG. 10 and implementation 1100 in FIG. 11, the first set of weights 1222 for implementation 1200 comprise an alternative representation generator 1224 that processes the input base sequence 1202 to generate an alternative representation of the input base sequence 1242. However, in contrast to implementations wherein the weights are trained end-to-end, the alternative sequence representation output 1242 from the alternative representation generator 1224 is mapped to an input 1206 for a succeeding model configured as a biological quantities output sequence generator 1228 which is used to train the second set of weights 1226 to generate a plurality of biological quantities output sequences 1246.

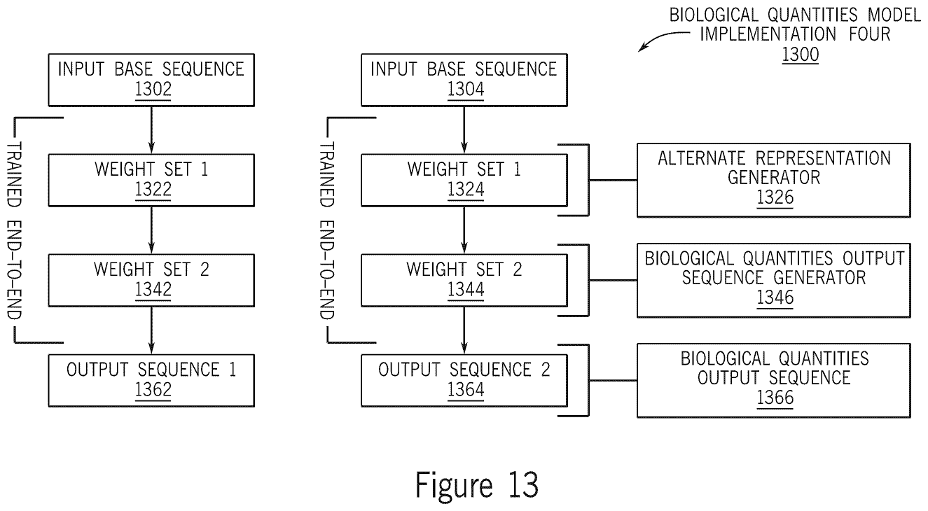

FIG. 13 is a flow diagram of one implementation 1300 of the technology disclosed in which the biological quantities model 124 generates one biological quantities output sequence 1362 from an input base sequence 1302 via a first set of weights 1322 and a second set of weights 1342 which are trained end-to-end and retrained to generate succeeding biological quantities output sequences on a singular basis. In the given example illustrated in FIG. 13, the biological quantities model 124 is trained on the first weight set 1322 and second weight set 1342 to generate the first output sequence 1362 in the plurality of biological quantities output sequences 1366. Succeeding the generation of the first output sequence, the biological quantities model 124 is retrained end-to-end on a first weight set 1324 and a second weight set 1344 to process an input base sequence 1304 and generate a second output sequence 1364. In the retraining process, the input base sequence 1304 is the same as the input base sequence 1302, the first weight set 1324 is the same as the first weight set 1322, and the second weight set 1344 is the same as the second weight set 1362. However, the second output sequence 1364 is a different biological quantities output sequence than the first biological quantities output sequence. For the total plurality of biological quantities output sequences 1366, the first and second weight are retrained end-to-end to produce each subsequent biological quantities output sequence.

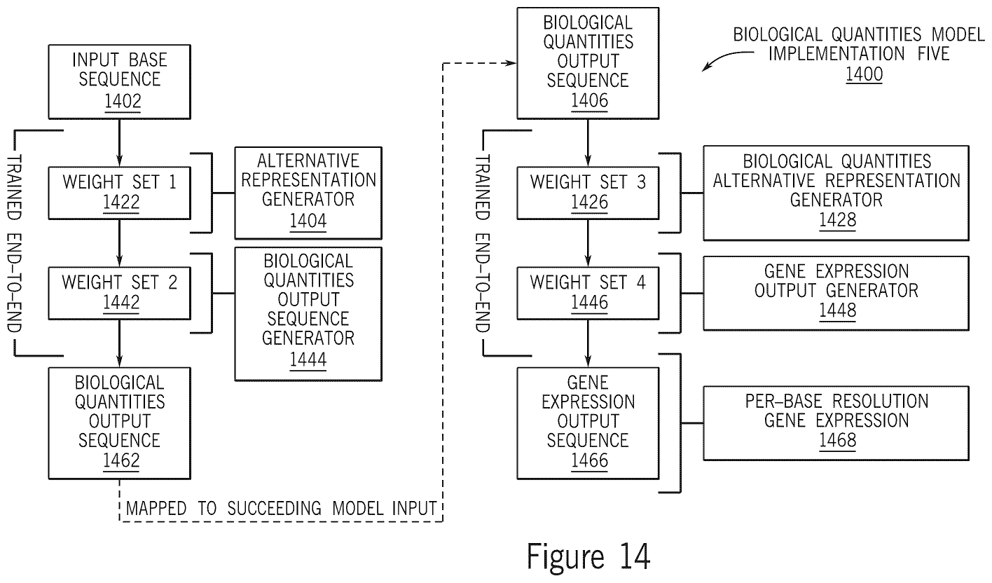

FIG. 14 is a flow diagram of one implementation 1400 of the technology disclosed in which the biological quantities model 124 comprises a first set of weights 1422 and second set of weights 1442 which are trained end-to-end to generate a plurality of biological quantities output sequences 1462 from an input base sequence, and a third and fourth set of weights 1426 and 1446 which are trained end-to-end to generate a gene expression output sequence 1466 from the input plurality of biological quantities output sequences 1406. The generation of biological quantities output sequences 1426 from an input base sequence 1402 is similar to implementation 1000 in FIG. 10 and implementation 1100 in FIG. 11 where the input base sequence 1402 is processed by a first set of weights 1422 configured as an alternative representation generator 1404 and a second set of weights 1442 as a biological quantities output sequence generator 1444 to produce a plurality of biological quantities output sequences 1462. The plurality of output sequences 1462 as an output of the second set of weights 1442 is then mapped to an input 1406 for a succeeding model configured to generate a gene expression output sequence 1466. The input biological quantities output sequences 1406 are processed by a third set of weights 1426 and fourth set of weights 1446 which are trained end-to-end. The third set of weights 1426 are configured as a biological quantities alternative representation generator 1428 and the fourth set of weights is configured as a gene expression output generator 1448. The resulting gene expression output sequence 1466 is a measure of base resolution gene expression 1468.

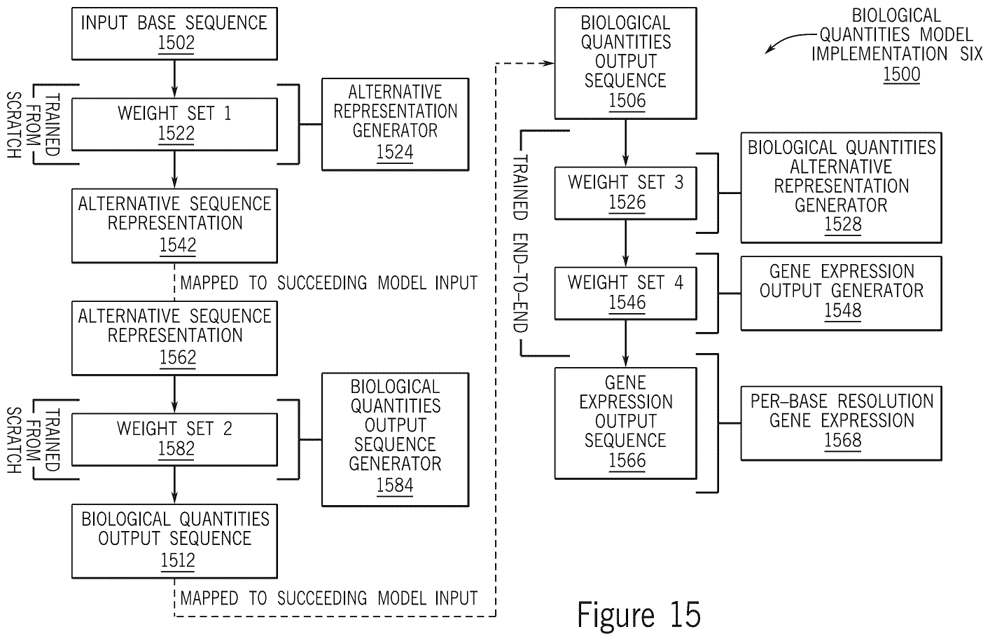

FIG. 15 is a flow diagram of one implementation 1500 of the technology disclosed in which the biological quantities model 124 comprises a first set of weights 1522 that generates an alternative representation of the input base sequence 1542, a second set of weights 1582 that is trained from scratch to generate a plurality of biological quantities output sequences 1512 from the alternative representation of the input base sequence 1562, and a third and fourth set of weights 1124 and 1546 which are trained end-to-end to generate a gene expression output sequence 1566 from the input plurality of biological quantities output sequences 1506. The generation of biological quantities output sequences 1512 from an input base sequence 1502 is similar to implementation 1200 in FIG. 12 where the biological quantities model 124 comprises a first set of weights 1522 that generates an alternate representation of the input base sequence 1542 and a second set of weights 1582 that is trained from scratch to generate a plurality of biological quantities output sequences 1512 from the alternate representation of the input base sequence 1562.

As in implementations 1000 from FIG. 10, 1100 from FIG. 11, 1200 from FIG. 12, 1300 from FIGS. 13, and 1400 from FIG. 14, the first weight set 1522 is configured as an alternative representation generator 1524 and the second set of weights 1582 is configured as a biological quantities output sequence generator 1584. The plurality of output sequences 1512 as an output of the second set of weights 1582 is then mapped to an input 1506 for a succeeding model configured to generate a gene expression output sequence 1566. The input biological quantities output sequences 1506 are processed by a third set of weights 1124 and fourth set of weights 1546 which are trained end-to-end. The third set of weights 1124 are configured as a biological quantities alternative representation generator 1528 and the fourth set of weights is configured as a gene expression output generator 1548. The resulting gene expression output sequence 1566 is a measure of base resolution gene expression 1568.

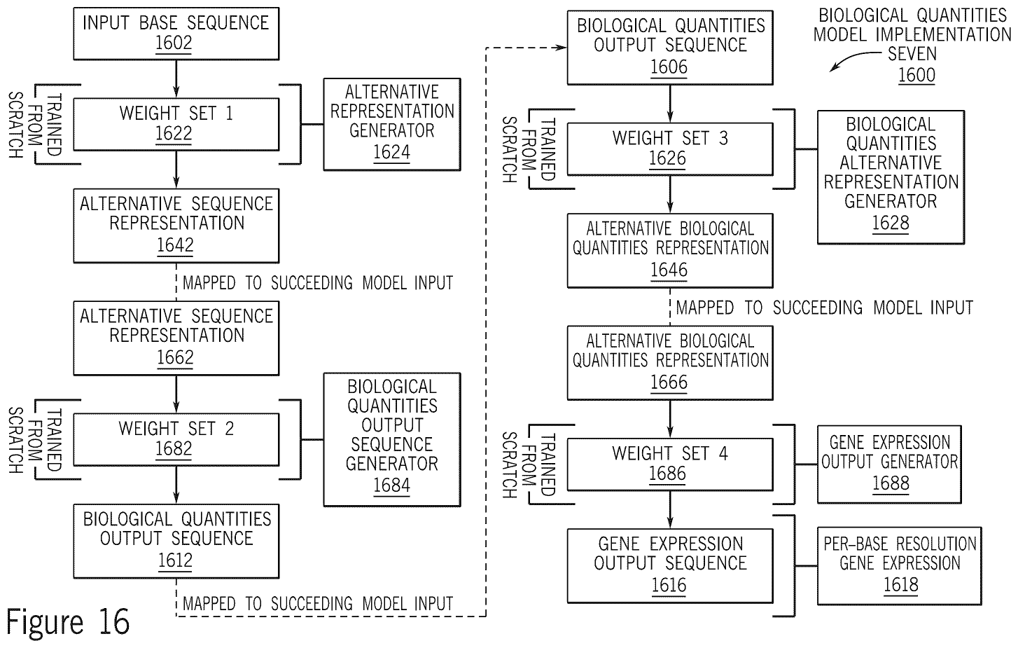

FIG. 16 is a flow diagram of one implementation 1600 of the technology disclosed in which the biological quantities model 124 comprises a first set of weights 1622 that generates an alternate representation of the input base sequence 1642, a second set of weights 1682 that is trained from scratch to generate a plurality of biological quantities output sequences 1612, a third set of weights 1124 that is trained from scratch to generate an alternative biological quantities representation 1646 from the plurality of biological quantities output sequences 1606, and a fourth set of weights 1686 that is trained from scratch to generate a gene expression output sequence 1616 from the alternative biological quantities representation 1666. The generation of biological quantities output sequences 1612 from an input base sequence 1602 is similar to implementation 1600 in FIG. 16 where the biological quantities model 124 comprises a first set of weights 1622 that generates an alternate representation of the input base sequence 1642 and a second set of weights 1682 that is trained from scratch to generate a plurality of biological quantities output sequences 1612 from the alternate representation of the input base sequence 1662.

As in implementations 1000 from FIG. 10, 1100 from FIG. 11, 1200 from FIG. 12, 1300 from FIG. 13, and 1400 from FIG. 14, and 1500 from FIG. 15, the first weight set 1622 is configured as an alternative representation generator 1624 and the second set of weights 1682 is configured as a biological quantities output sequence generator 1684. The plurality of biological quantities output sequences 1612 as an output of the second set of weights 1682 is then mapped to an input 1606 for a succeeding model configured to generate an alternate biological quantities representation 1646. The succeeding model is configured as a biological quantities alternative representation generator 1628 that comprises a third set of weights 1124 that is trained from scratch to produce an alternative biological quantities representation 1646. The alternate biological quantities representation output 1646 as an output of the third set of weights 1124 is then mapped to an input 1666 for a second succeeding model to generate a gene expression output sequence 1616. The second succeeding model is configured as a gene expression output generator 1688 that comprises a fourth weight set 1686 that is trained from scratch to generate a gene expression output sequence 1616. The resulting gene expression output sequence 1616 is a measure of base resolution gene expression 1618.

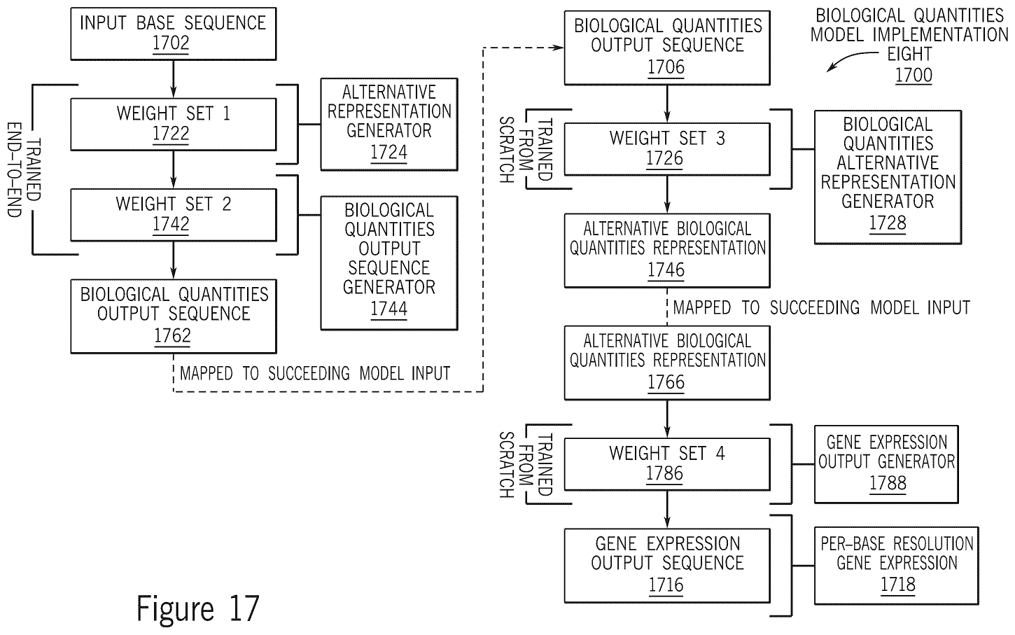

FIG. 17 is a flow diagram of one implementation 1700 of the technology disclosed in which the biological quantities model 124 comprises a first set of weights 1722 and second set of weights 1742 which can be trained end-to-end to generate a plurality of biological quantities output sequences 1762 from an input base sequence 1702. A third set of weights 1726 can be trained from scratch to generate an alternative biological quantities representation 1746 from the plurality of biological quantities output sequences 1706. A fourth set of weights 1786 can be trained from scratch to generate a gene expression output sequence 1716 from the alternative biological quantities representation 1766. A given per-base gene expression output in the gene expression output sequence 1716 for the given target base at the given position specifies a measure of gene expression level of the given target base at the given position. In one implementation, the gene expression level is measured in a per-base metric such as CAGE transcription start site (CTSS). In another implementation, the gene expression level is measured in a per-gene metric such as transcripts per million (TPM) or reads per kilobase of transcript (RPKM). In yet another implementation, the gene expression level is measured in a per-gene metric such as fragments per kilobase million (FPKM).

The generation of biological quantities output sequences 1762 from an input base sequence 1702 is similar to implementation 1000 in FIG. 10 and implementation 1100 in FIG. 11 where the input base sequence 1702 can be processed by a first set of weights 1722 applied by an alternative representation generator 1724, and a second set of weights 1742 applied by a biological quantities output sequence generator 1744 to produce a plurality of biological quantities output sequences 1762.

The plurality of biological quantities output sequences 1762, as an output of the second set of weights 1742, can then mapped to an input 1706 for a succeeding model configured to generate an alternate biological quantities representation 1746. The succeeding model can configured as a biological quantities alternative representation generator 1728 that comprises a third set of weights 1726 that is trained from scratch to produce an alternative biological quantities representation 1746. The alternate biological quantities representation output 1746 as an output of the third set of weights 1726 can then mapped to an input 1766 for a second succeeding model to generate a gene expression output sequence 1716. The second succeeding model can be configured as a gene expression output generator 1788 that comprises a fourth weight set 1786 that is trained from scratch to generate a gene expression output sequence 1716. The resulting gene expression output sequence 1716 is a measure of base resolution gene expression 1718.

### Application of Transfer Learning to the Biological Quantities Model

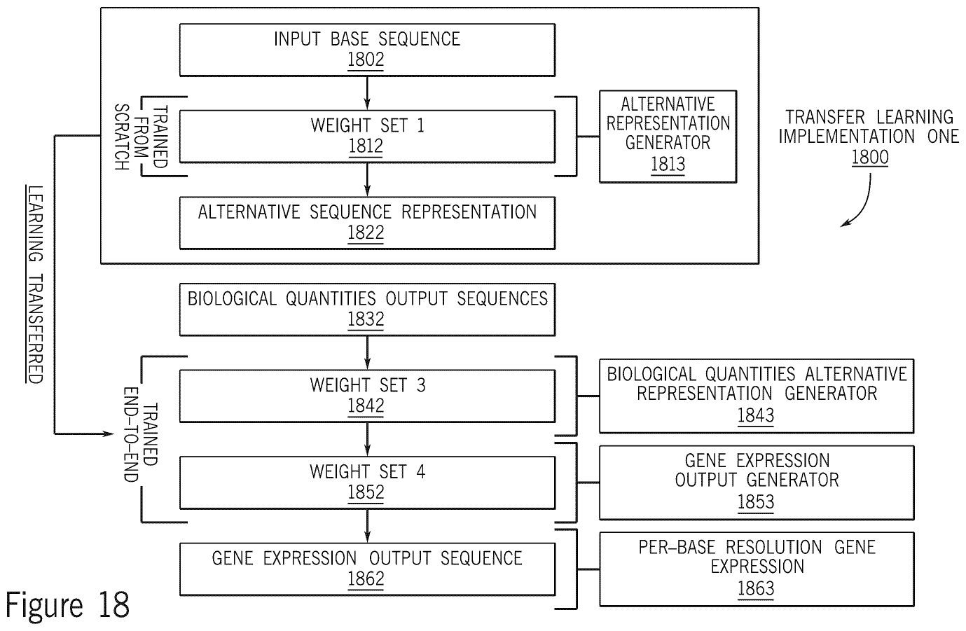

FIG. 18 is a flow diagram of one implementation 1800 of the technology disclosed in which the biological quantities model 124 comprises a first set of weights 1812 that is trained to generate an alternative sequence representation 1822 from an input base sequence 1802, and then retrained as a substitute of the third set of weights 1842 to generate an alternative biological quantities representation from the plurality of biological quantities output sequences 1832, and a fourth set of weights 1852 that is trained end-to-end with the substituted first set of weights to generate a gene expression output sequence 1862 from the biological quantities output sequences 1832. The optimized weight scalar values for weight set one 1812 that are learned from the alternative representation generator 1813 can be transferred as substitute scalar values for each weight within the third set of weights 1842. As in implementation 1400 in FIG. 14 and implementation 1500 in FIG. 15, the biological quantities alternative representation generator 1843 comprises a third set of weights 1842 that is trained end-to-end with the fourth set of weights 1852 that comprise the gene expression output generator 1863. The resulting gene expression output sequence 1862 is a measure of base resolution gene expression 1863.

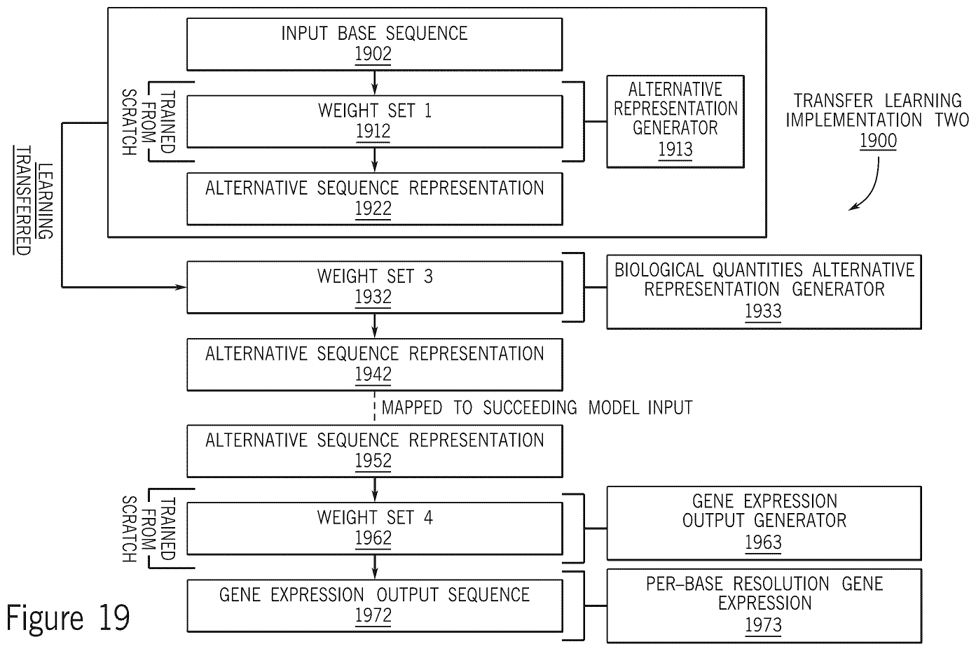

FIG. 19 is a flow diagram of one implementation 1900 of the technology disclosed in which the biological quantities model 124 comprises a first set of weights 1912 that is trained to generate an alternative sequence representation 1922 from an input base sequence 1902, and then retrained as a substitute of the third set of weights 1932 to generate an alternative biological quantities representation 1942 from the alternative sequence representation 1922, and a fourth set of weights 1962 that is trained from scratch to generate a gene expression output sequence 1972 from the alternative biological quantities representation 1952. The optimized weight scalar values for weight set one 1912 that are learned from the alternative representation generator 1913 can be transferred as substitute scalar values for each weight within the third set of weights 1932.

The alternate biological quantities representation output 1942 as an output of the third set of weights 1932 is then mapped to an input 1952 for a succeeding model to generate a gene expression output sequence 1716. As in implementation 1600 from FIG. 16 and implementation 1700 from FIG. 17, the succeeding model is configured as a gene expression output generator 1963 that comprises a fourth weight set 1962 that is trained from scratch to generate a gene expression output sequence 1972. The resulting gene expression output sequence 1972 is a measure of base resolution gene expression 1973.

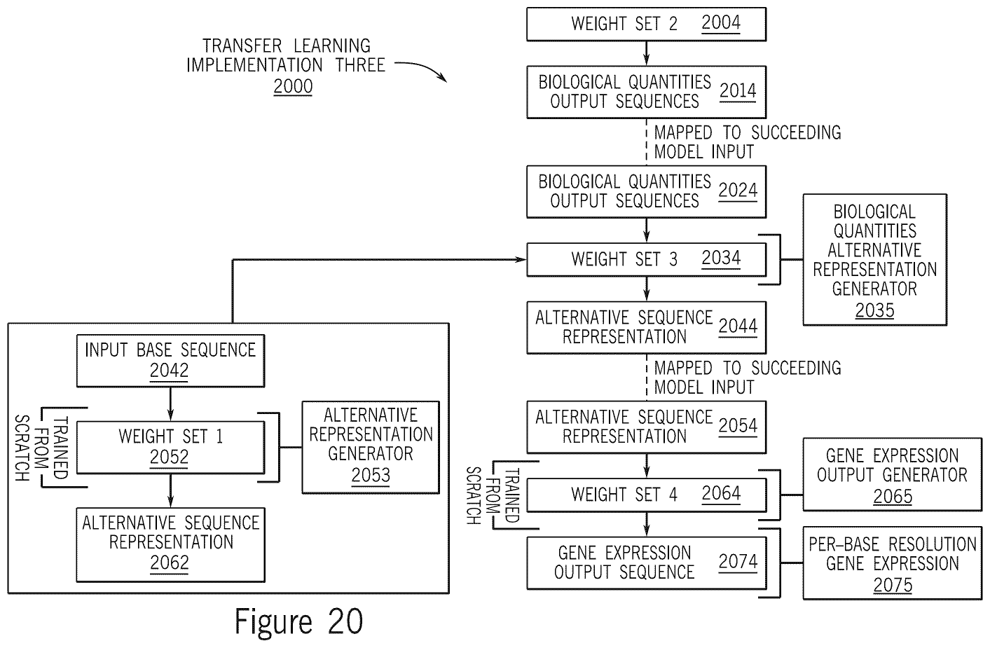

FIG. 20 is a flow diagram of one implementation 2000 of the technology disclosed in which the biological quantities model 124 comprises a first set of weights 2052 that is trained to generate an alternative sequence representation 2062 from an input base sequence 2042, a second set of weights 2004 that is trained to generate a plurality of biological quantities output sequences 2014, the retrained first set of weights 2052 used as a substitute of the third set of weights 2034 to generate an alternative biological quantities representation 2044 from the plurality of biological quantities output sequences, and a fourth set of weights 2064 that is trained from scratch to generate a gene expression output sequence 2074 from the alternative biological quantities representation 2054. As in implementation 1900 from FIG. 19, optimized weight scalar values for weight set one 2052 that are learned from the alternative representation generator 2053 can be transferred as substitute scalar values for each weight within the third set of weights 2034.

The second set of weights 2004 is configured to generate a plurality of biological quantities output sequences 2014. As in implementation 1400 from FIG. 14, implementation 1500 from FIG. 15, implementation 1600 from FIG. 16, and implementation 1700 from FIG. 17, of biological quantities output sequences 2014 is mapped to the input of a succeeding model configured as a biological quantities alternative representation generator 2035 that comprises a third weight set 2034. In implementation 2000, the scalar values for the third set of weights 2034 are substituted from the optimized scalar values from the trained first set of weights 2052. As in implementation 1600 from FIG. 16 and implementation 1700 from FIG. 17, the alternate biological quantities representation output 2044 as an output of the third set of weights 2034 is then mapped to an input 2044 for a succeeding model to generate a gene expression output sequence 2074. The succeeding model is configured as a gene expression output generator 2065 that comprises a fourth weight set 2064 that is trained from scratch to generate a gene expression output sequence 2074. The resulting gene expression output sequence 2074 is a measure of base resolution gene expression 2075.

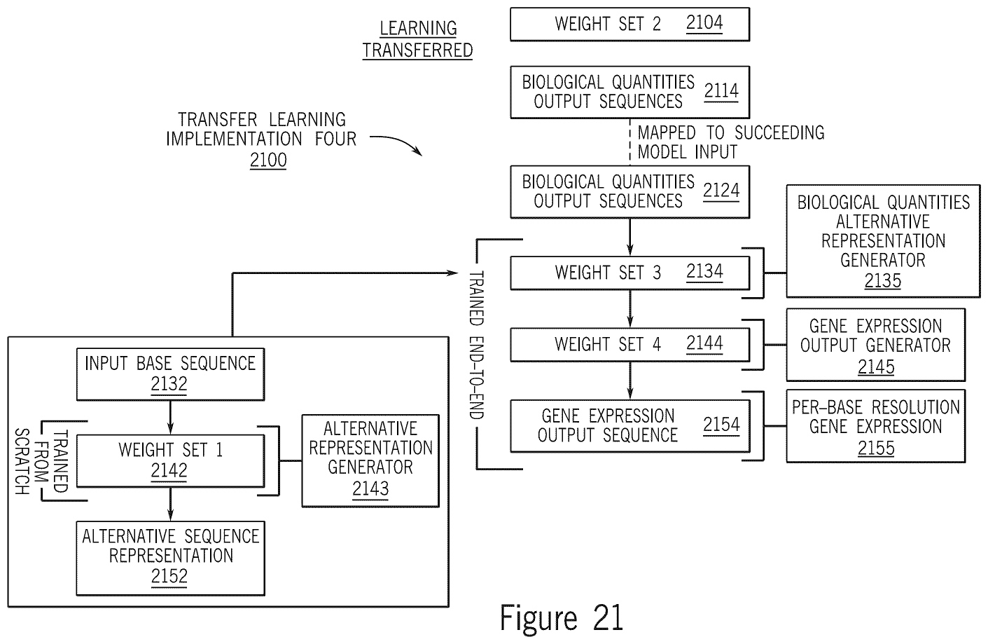

FIG. 21 is a flow diagram of one implementation 2100 of the technology disclosed in which the biological quantities model 124 comprises a first set of weights 2142 that is trained to generate an alternative sequence representation 2152 from an input base sequence 2132, a second set of weights 2104 that is trained to generate a plurality of biological quantities output sequences 2114 from the alternate representation of the input base, the retrained first set of weights 2142 used as a substitute of the third set of weights 2134 to generate an alternative biological quantities representation from the plurality of biological quantities output sequences 2124, and a fourth set of weights 2144 that is trained end-to-end with the substituted first set of weights 2142 to generate a gene expression output sequence 2154 from the alternative biological quantities representation. As in implementation 1900 from FIG. 19 and implementation 2000 from FIG. 20, optimized weight scalar values for weight set one 2142 that are learned from the alternative representation generator 2152 can be transferred as substitute scalar values for each weight within the third set of weights 2134.

As in implementation 2000 from FIG. 20, the second set of weights 2104 is configured to generate a plurality of biological quantities output sequences 2114. As in implementation 1400 from FIG. 14, implementation 1500 from FIG. 15, implementation 1600 from FIG. 16, and implementation 1700 from FIG. 17, of biological quantities output sequences 2114 is mapped to the input of a succeeding model configured as a biological quantities alternative representation generator 2135 that comprises a third weight set 2134. As in implementation 2000 from FIG. 20, the scalar values for the third set of weights 2134 are substituted from the optimized scalar values from the trained first set of weights 2142. As in implementation 1400 in FIG. 14, implementation 1500 in FIG. 15, and implementation 1800 in FIG. 18, the biological quantities alternative representation generator 2135 comprises a third set of weights 2134 that is trained end-to-end with the fourth set of weights 2144 that comprise the gene expression output generator 2145. The resulting gene expression output sequence 2154 is a measure of base resolution gene expression 2155.

### Pathogenicity Prediction at Base Resolution

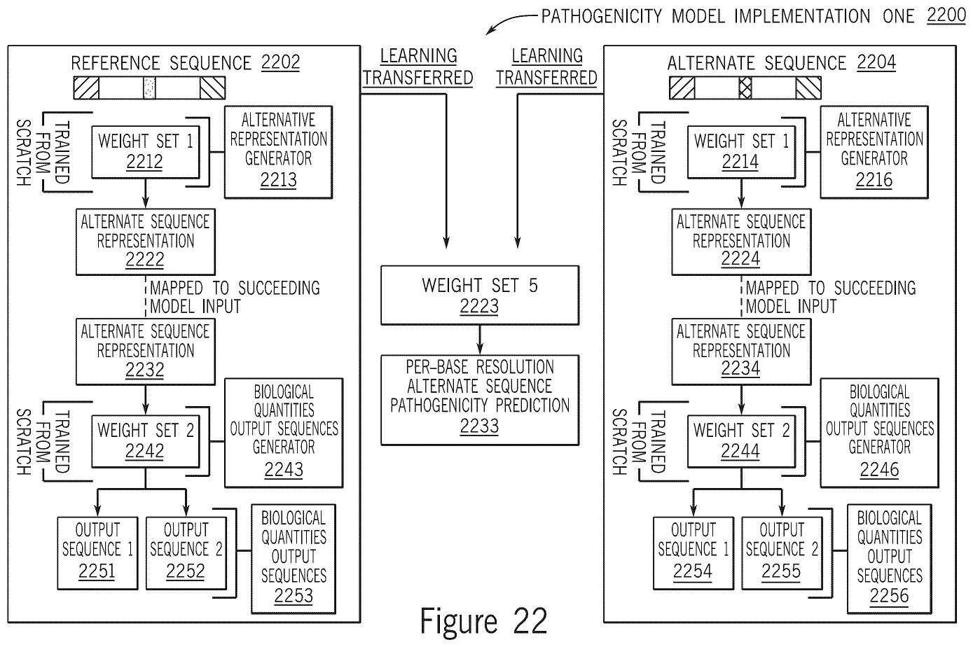

FIG. 22 is a flow diagram of one implementation 2200 of the technology disclosed in which the biological quantities model 124 is further configured to comprise a pathogenicity prediction logic that compares the reference biological quantities output sequences 2253 and the alternate biological quantities output sequences 2256 at base resolution wherein a fifth set of weights 2223 generates an alternate sequence pathogenicity prediction 2233 from the plurality of biological quantities output sequences 2253 and 2256 wherein the first set of weights 2212 and 2214 and second set of weights 2242 and 2244 are each trained from scratch. As in implementation 1200 in FIG. 12, implementation 1500 in FIG. 15, and implementation 1600 in FIG. 16, the first set of weights 2212 for implementation 2200 comprise an alternative representation generator 2213 that processes the reference input base sequence 2202 to generate an alternative representation 2222 of the reference input base sequence 2202. The alternative sequence representation output 2222 from the alternative representation generator 2213 is mapped to an input 2232 for a succeeding model configured as a biological quantities output sequence generator 2243 which is used to train the second set of weights 2242 to generate a plurality of biological quantities output sequences 2253.

In parallel, the first set of weights 2214 for implementation 2200 comprise an alternative representation generator 2216 that processes the alternate input base sequence 2204 to generate an alternative representation 2224 of the alternate input base sequence 2204. The alternative sequence representation output 2224 from the alternative representation generator 2216 is mapped to an input 2234 for a succeeding model configured as a biological quantities output sequence generator 2246 which is used to train the second set of weights 2244 to generate a plurality of biological quantities output sequences 2256. The optimized scalar values of the weights for each respective biological quantities model 124 for the reference sequence 2202 and the alternate sequence 2204 can be transferred to a fifth set of weights 2223 that generates a base resolution alternate sequence pathogenicity prediction 2233.

In one implementation, the pathogenicity prediction can be a score between zero and one, where zero represents absolute benignness and one represents absolute pathogenicity. In other implementations, a cutoff can be used, such as a pathogenicity score above five, for example, can be considered pathogenic, and below five can be considered benign.

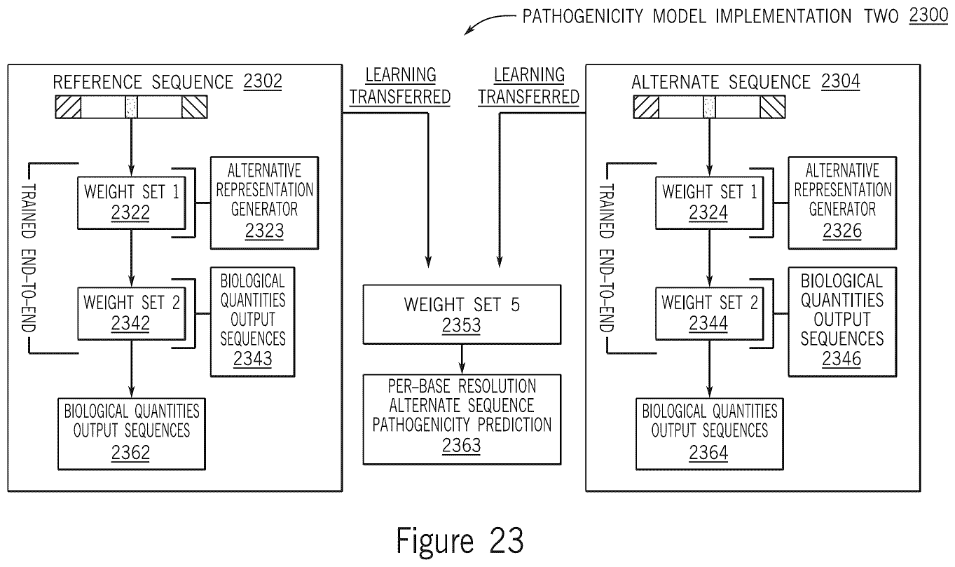

FIG. 23 is a flow diagram of one implementation 2300 of the technology disclosed in which the biological quantities model 124 is further configured to comprise a pathogenicity prediction logic that compares the reference biological quantities output sequences 2362 and the alternate biological quantities output sequences 2364 at base resolution wherein a fifth set of weights 2353 generates an alternate sequence pathogenicity prediction 2363 from the plurality of biological quantities output sequences 2362 and 2364 wherein the first set of weights 2322 and 2324 and second set of weights 2342 and 2344 are trained end-to-end. As in implementation 1100 in FIG. 11, 1200 in FIG. 12, 1300 in FIG. 13, 1400 in FIGS. 14, and 1700 in FIG. 17, the reference input base sequence 2302 is processed by a first set of weights 2322 configured as an alternative representation generator 2323 and a second set of weights 2342 as a biological quantities output sequence generator 2343 to generate a plurality of biological quantities output sequences 2362. The first set of weights 2322 and second set of weights 2342 are trained end-to-end.

In parallel, the first set of weights 2324 process the alternate input base sequence 2304 comprise an alternate representation generator 2326 and the second set of weights 2344 comprise a biological quantities output sequence generator 2346 to generate a plurality of biological quantities output sequences 2364 in the same fashion as the plurality biological quantities output sequences 2362 are generated from the reference input base sequence 2302. The optimized scalar values of the weights for each respective biological quantities model 124 for the reference sequence 2302 and the alternate sequence 2304 can be transferred to a fifth set of weights 2353 that generates a base resolution alternate sequence pathogenicity prediction 2363.

### Biological Quantities Output Sequence Data

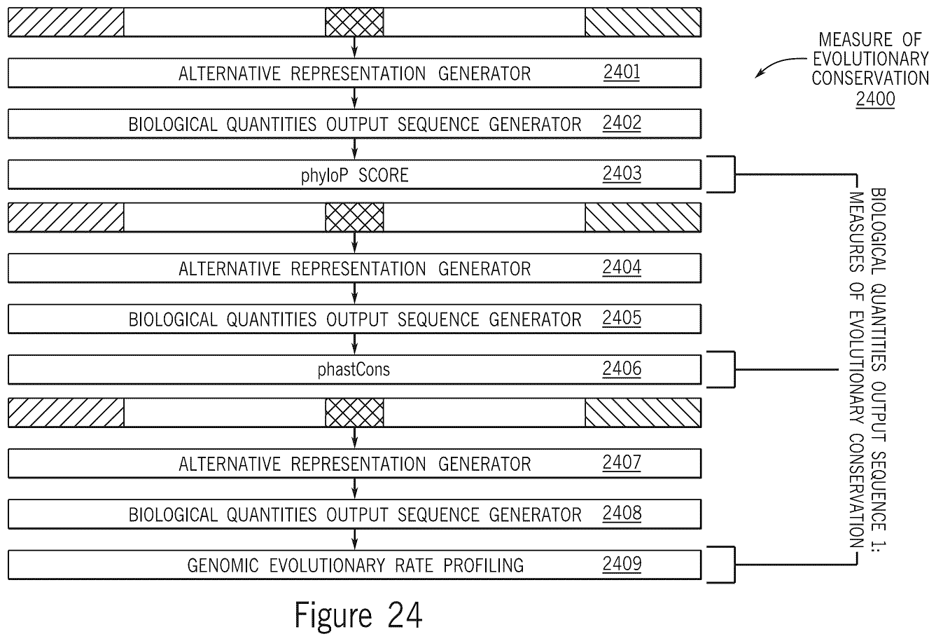

FIG. 24 is a schematic of the measures of evolutionary conservation 2400 that can be generated from the biological quantities model 124 from the input base sequence as a value for the first biological quantities output sequence. After an input base sequence is processed by an alternative representation generator 2401, the biological quantities output sequence generator 2402 can generate a first biological quantities output sequence in the form of a phyloP score 2403. After an input base sequence is processed by an alternative representation generator 2404, the biological quantities output sequence generator 2405 can generate a first biological quantities output sequence in the form of a phastCons value 2406. After an input base sequence is processed by an alternative representation generator 2407, the biological quantities output sequence generator 2408 can generate a first biological quantities output sequence in the form of a phastCons value 2409.

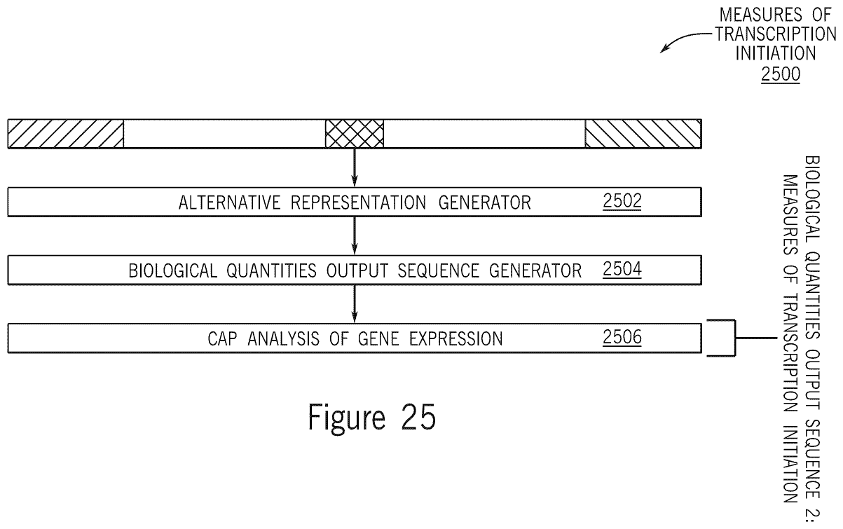

FIG. 25 is a schematic of the measure of transcription initiation 2500 that can be generated from the biological quantities model 124 from the input base sequence as a value for the second biological quantities output sequence. After an input base sequence is processed by an alternative representation generator 2502, the biological quantities output sequence generator 2504 can generate a second biological quantities output sequence in the form of a cap analysis of gene expression (CAGE) value 2506.

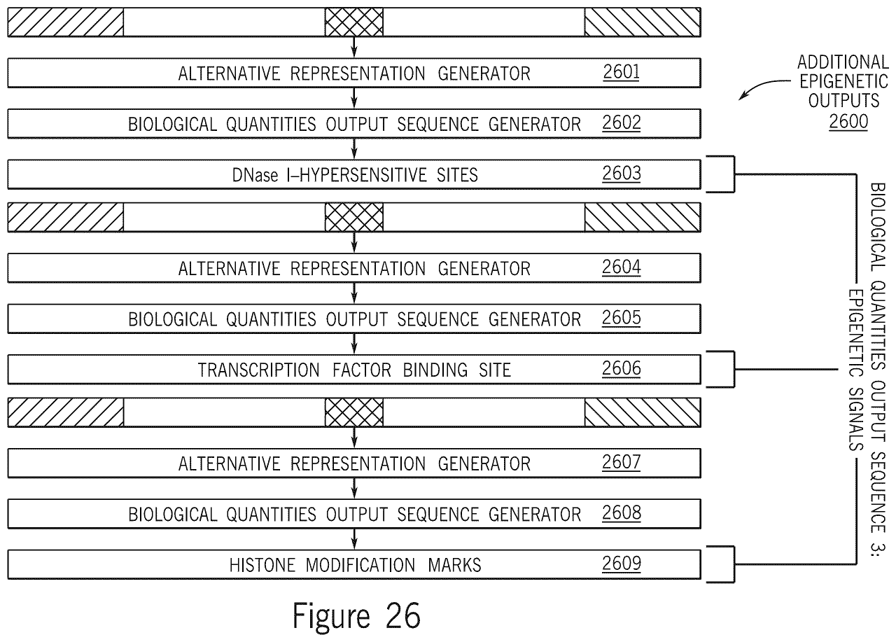

FIG. 26 is a schematic of the epigenetic signals 2600 that can be generated from the biological quantities model 124 from the input base sequence as a value for the third biological quantities output sequence. After an input base sequence is processed by an alternative representation generator 2601, the biological quantities output sequence generator 2602 can generate a first biological quantities output sequence in the form of a DNase I-hypersensitive site prediction output 2603. After an input base sequence is processed by an alternative representation generator 2604, the biological quantities output sequence generator 2605 can generate a first biological quantities output sequence in the form of a transcription factor binding site prediction output 2606. After an input base sequence is processed by an alternative representation generator 2607, the biological quantities output sequence generator 2608 can generate a first biological quantities output sequence in the form of a histone modification mark prediction output 2609.

### Gene Expression Classification Models

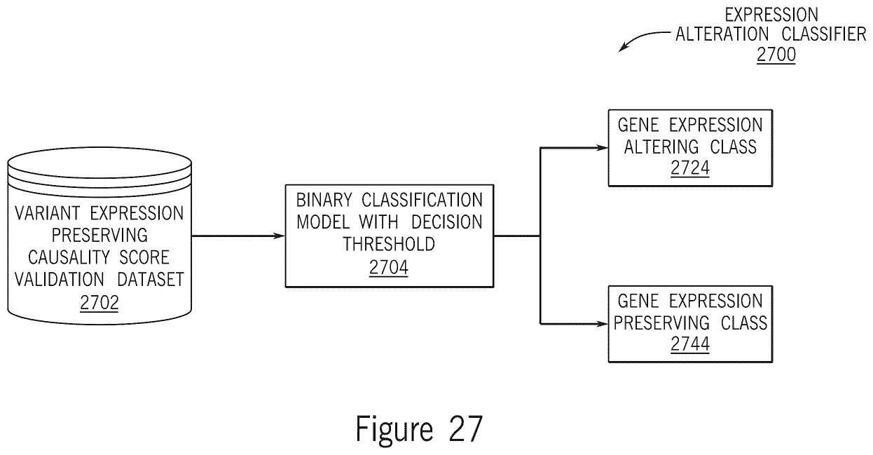

FIG. 27 is a flow diagram of one implementation of the technology disclosed in which an expression alteration classifier 2700 is configured to predict the effect of a variant on gene expression. A variant expression-preserving causality score validation dataset 2702 is used to generate a ground truth bifurcation of the set of variants for a binary classification model 2704 with a specified decision threshold value (e.g., p-value cutoff less than 0.01, 0.0001, or 1e-14) that can be employed to classify a variant into a gene expression altering class 2724, i.e., variants that change gene expression, or a gene expression preserving class 2744 i.e., variants that do not change gene expression. The classification of a variant is learned from an assigned causality score that specifies a statistically unconfounded likelihood of altering gene expression.

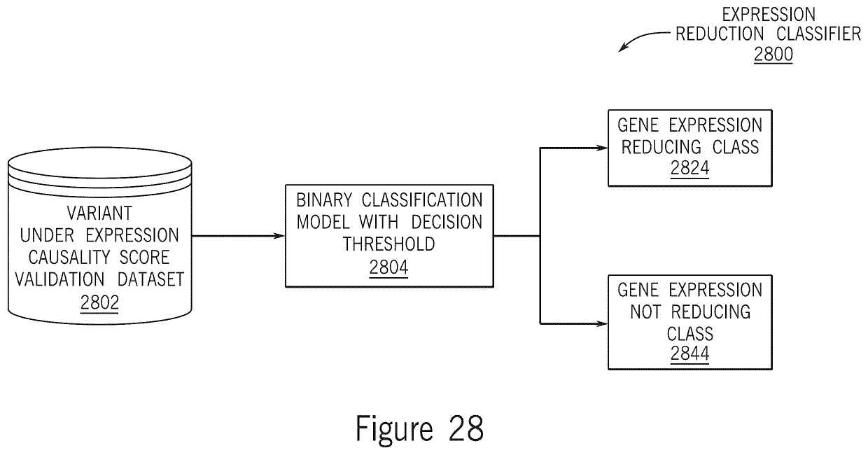

FIG. 28 is a flow diagram of one implementation of the technology disclosed in which the expression alteration classifier is further configured as an expression reduction classifier 2800 to predict if a variant reduces gene expression or does not reduce gene expression. A variant under expression causality score validation set is used to generate an under expression ground truth bifurcation of the set of variants for a binary classification model 2804 with a specified decision threshold value (e.g., p-value cutoff less than 0.01, 0.0001, or 1e-14) that can be employed to classify a variant into a gene expression reducing class 2824, i.e., variants that reduce gene expression, or a gene expression not reducing class 2844, i.e., variants that do not reduce gene expression. The classification of a variant is learned from an assigned under expression causality score that specifies a statistically unconfounded likelihood of reducing gene expression.

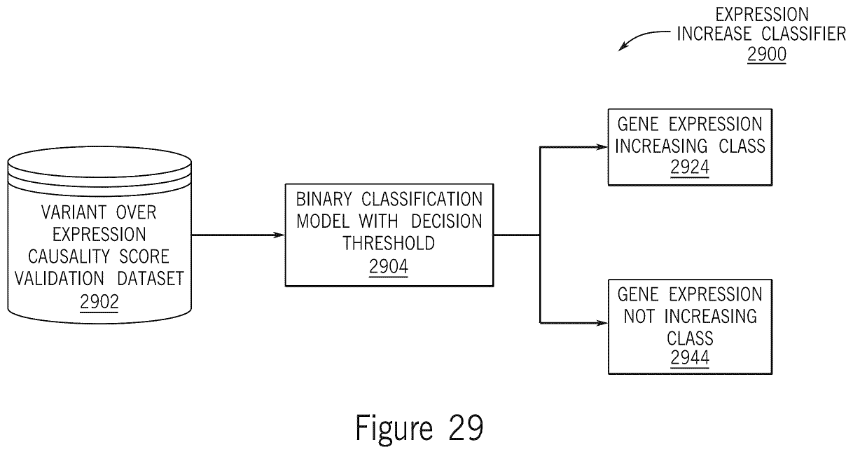

FIG. 29 is a flow diagram of one implementation of the technology disclosed in which the expression alteration classifier is further configured as an expression increase classifier 2900 to predict if a variant increases gene expression or does not increase gene expression. A variant over expression causality score validation dataset 2902 is used to generate an over expression ground truth bifurcation of the set of variants for a binary classification model 2904 with a specified decision threshold value (e.g., p-value cutoff less than 0.01, 0.0001, or 1e-14) that can be employed to classify a variant into a gene expression reducing class 2924 or a gene expression not reducing class 2944. The classification of a variant is learned from an assigned over expression causality score that specifies a statistically unconfounded likelihood of increasing gene expression.

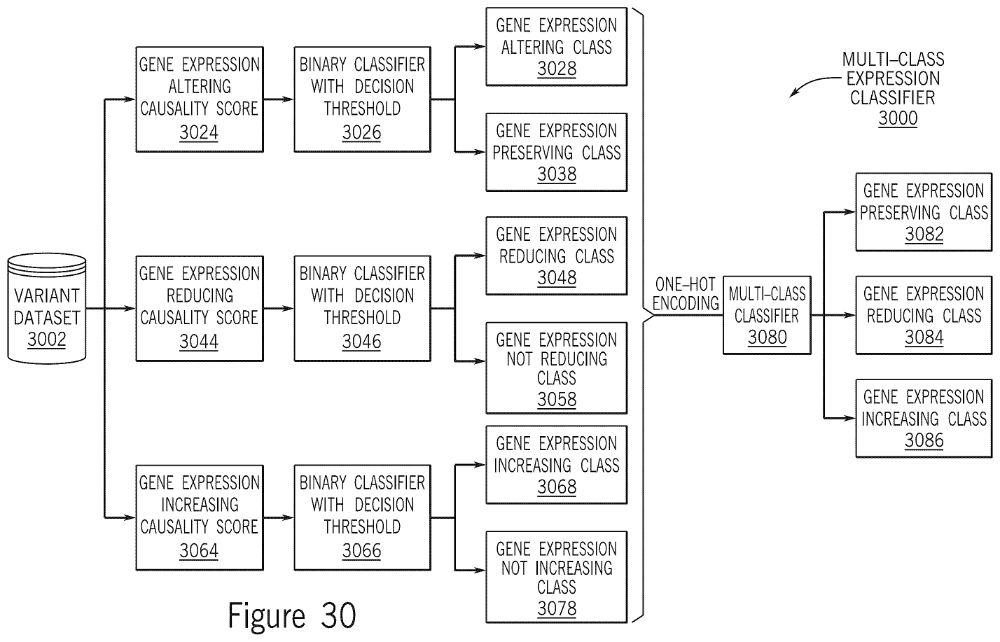

FIG. 30 is a flow diagram of one implementation of the technology disclosed in which the expression alteration classifier is further configured into a multi-class expression classifier 3000 that predicts if a variant preserves gene expression, reduces gene expression, or increases gene expression. A variant dataset is processed by a system 3000 in which a first performance measure is generated for the comparison of the inferred bifurcation against the ground truth bifurcation through a binary classifier with decision threshold 3026 that generates an output corresponding to the gene expression altering class 3028 or the gene expression preserving class 3038 from a gene expression altering causality score 3024, a second performance measure is generated for the comparison of the inferred bifurcation against the ground truth bifurcation through a binary classifier with decision threshold 3046 that generates an output corresponding to the gene expression reducing class 3048 or the gene expression not reducing class 3058 from a gene expression reducing causality score 3044, and a third performance measure is generated for the comparison of the inferred bifurcation against the ground truth bifurcation through a binary classifier with decision threshold 3066 that generates an output corresponding to the gene expression increasing class 3068 or the gene expression not increasing class 3078 from a gene expression increasing causality score 3064.

The system 3000 is configured to require the first, second, and third inferred bifurcations to classify a same number of variants in the gene altering class thereby making the first, second, and third performance measures comparable to each other. The system 3000 is further configured to compare respective performances of the first binary classifier with decision threshold 3026, the second binary classifier 3046, and the third binary classifier 3066 on the validation data 3002 based on a comparison of the first, second, and third performance measures. The decision thresholds for the first binary classifier with decision threshold 3026, the second binary classifier 3046, and the third binary classifier 3066 may be the same. The decision thresholds for the first binary classifier with decision threshold 3026, the second binary classifier 3046, and the third binary classifier 3066 may be different.

The system 3000 is further configured to generate a ground truth trifurcation and an inferred trifurcation of the set of variants 3002 into a gene expression preserving class 3082, a gene expression reducing class 3084, and a gene expression increasing class 3086 from a multiclass classifier 3080 that processes one-hot encoded vectors containing the ground truth bifurcations and inferred bifurcations from the first binary classifier with decision threshold 3026, the second binary classifier 3046, and the third binary classifier 3066.

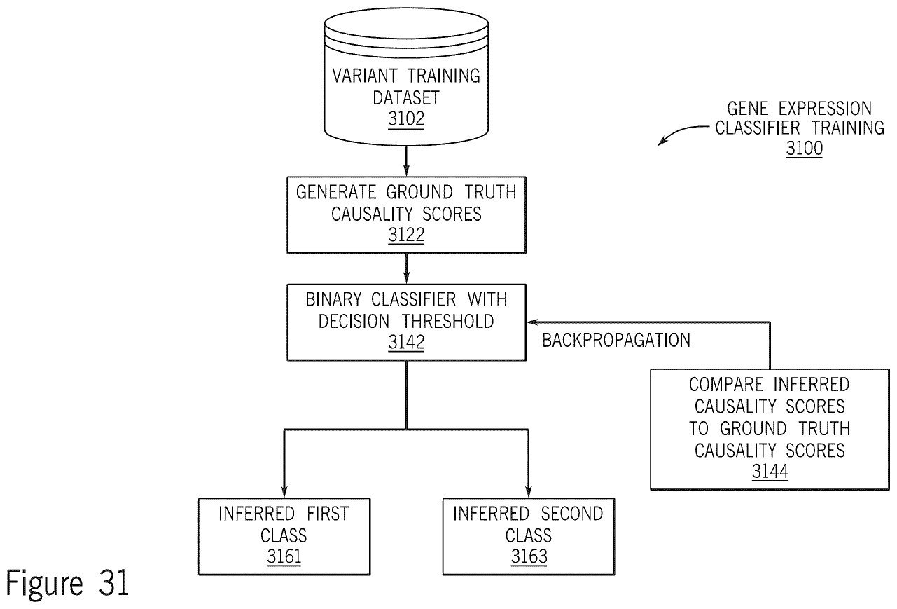

FIG. 31 is a flow diagram of one implementation of the technology disclosed in which gene expression classifier training 3100 is employed for the comparison of the ground truth causality scores to the inferred causality scores 3144. A set of ground truth causality scores 3122 is generated for the variant training dataset 3102. A binary classifier with decision threshold 3142 processes the variant training dataset 3102 to generate inferred causality scores classified into an inferred first class 3161 and an inferred second class 3163. The gene expression classifier training protocol 3100 performs backpropagation on the weights of the binary classifier 3142 for a number of iterations to optimize a loss function.

### Computer System

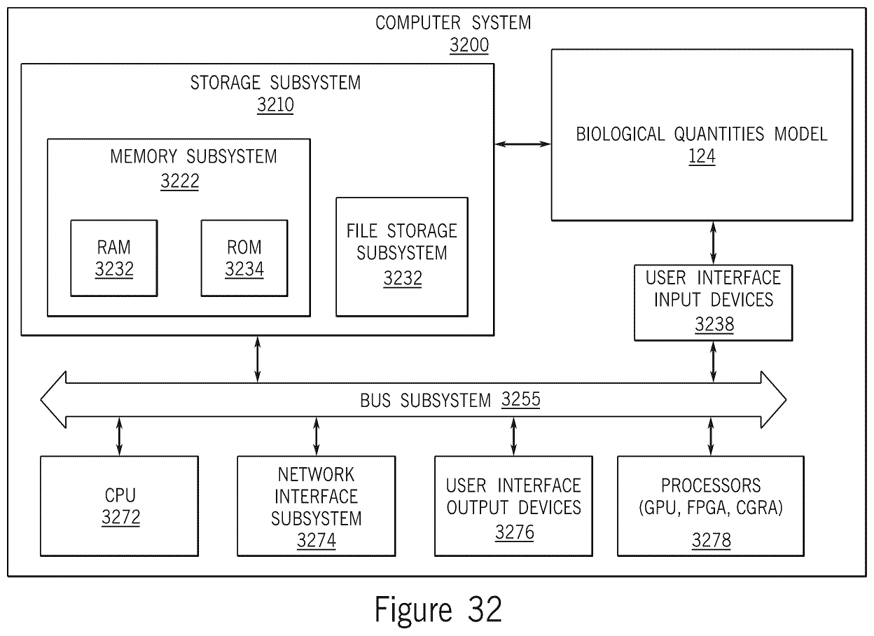

FIG. 32 shows an example computer system 3200 that can be used to implement the technology disclosed. Computer system 3200 includes at least one central processing unit (CPU) 3272 that communicates with a number of peripheral devices via bus subsystem 3255. These peripheral devices can include a storage subsystem 3210 including, for example, memory devices and a file storage subsystem 3232, user interface input devices 3238, user interface output devices 3276, and a network interface subsystem 3274. The input and output devices allow user interaction with computer system 3200. Network interface subsystem 3274 provides an interface to outside networks, including an interface to corresponding interface devices in other computer systems.

In one implementation, the biological quantities model 124 is communicably linked to the storage subsystem 3210 and the user interface input devices 3238.

User interface input devices 3238 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system 3200.

User interface output devices 3276 can include a display subsystem, a printer, a fax machine, or non-visual displays such as audio output devices. The display subsystem can include an LED display, a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system 3200 to the user or to another machine or computer system.

Storage subsystem 3210 stores programming and data constructs that provide the functionality of some or all of the modules and methods described herein. These software modules are generally executed by processors 3278.

Processors 3278 can be graphics processing units (GPUs), field-programmable gate arrays (FPGAs), application-specific integrated circuits (ASICs), and/or coarse-grained reconfigurable architectures (CGRAs). Processors 3278 can be hosted by a deep learning cloud platform such as Google Cloud Platform™, Xilinx™, and Cirrascale™ Examples of processors 3278 include Google's Tensor Processing Unit (TPU)™, rackmount solutions like GX4 Rackmount Series™, GX32 Rackmount Series™, NVIDIA DGX-1, Microsoft' Stratix V FPGA™, Graphcore's Intelligent Processor Unit (IPU)™, Qualcomm's Zeroth Platform™ with Snapdragon processors™, NVIDIA's Volta™, NVIDIA's DRIVE PX™, NVIDIA's JETSON TX1/TX2 MODULE™, Intel's Nirvana™, Movidius VPU™, Fujitsu DPI™, ARM's DynamicIQ™, IBM TrueNorth™, Lambda GPU Server with Testa V100s™, and others.

Memory subsystem 3222 used in the storage subsystem 3210 can include a number of memories including a main random access memory (RAM) 3232 for storage of instructions and data during program execution and a read only memory (ROM) 3234 in which fixed instructions are stored. A file storage subsystem 3232 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem 3232 in the storage subsystem 3210, or in other machines accessible by the processor.

Bus subsystem 3255 provides a mechanism for letting the various components and subsystems of computer system 3200 communicate with each other as intended. Although bus subsystem 3255 is shown schematically as a single bus, alternative implementations of the bus subsystem can use multiple busses.

Computer system 3200 itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system 3200 depicted in FIG. 32 is intended only as a specific example for purposes of illustrating the preferred implementations of the present invention. Many other configurations of computer system 3200 are possible having more or less components than the computer system depicted in FIG. 32.

### Clauses

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections-these recitations are hereby incorporated forward by reference into each of the following implementations.

One or more implementations and clauses of the technology disclosed or elements thereof can be implemented in the form of a computer product, including a non-transitory computer readable storage medium with computer usable program code for performing the method steps indicated. Furthermore, one or more implementations and clauses of the technology disclosed or elements thereof can be implemented in the form of an apparatus including a memory and at least one processor that is coupled to the memory and operative to perform exemplary method steps. Yet further, in another aspect, one or more implementations and clauses of the technology disclosed or elements thereof can be implemented in the form of means for carrying out one or more of the method steps described herein; the means can include (i) hardware module(s), (ii) software module(s) executing on one or more hardware processors, or (iii) a combination of hardware and software modules; any of (i)-(iii) implement the specific techniques set forth herein, and the software modules are stored in a computer readable storage medium (or multiple such media).

The clauses described in this section can be combined as features. In the interest of conciseness, the combinations of features are not individually enumerated and are not repeated with each base set of features. The reader will understand how features identified in the clauses described in this section can readily be combined with sets of base features identified as implementations in other sections of this application. These clauses are not meant to be mutually exclusive, exhaustive, or restrictive; and the technology disclosed is not limited to these clauses but rather encompasses all possible combinations, modifications, and variations within the scope of the claimed technology and its equivalents.

Other implementations of the clauses described in this section can include a non-transitory computer readable storage medium storing instructions executable by a processor to perform any of the clauses described in this section. Yet another implementation of the clauses described in this section can include a system including memory and one or more processors operable to execute instructions, stored in the memory, to perform any of the clauses described in this section.

We disclose the following clauses:

### Clauses Set 1

1. An artificial intelligence-based system to detect changes in gene expression at base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a biological quantities model that processes the input base sequence and generates an alternative representation of the input base sequence; and
   a biological quantities output generation logic that processes the alternative representation of the input base sequence and generates a plurality of biological quantities output sequences,
      wherein a first biological quantities output sequence in the plurality of biological quantities output sequences includes a first respective per-base biological quantities outputs for the respective target bases in the target base sequence,
         wherein the first respective per-base biological quantities outputs specify respective measurements of evolutionary conservation of the respective target bases across a plurality of species, and
      wherein a second biological quantities output sequence in the plurality of biological quantities output sequences includes a second respective per-base biological quantities outputs for the respective target bases in the target base sequence,
         wherein the second respective per-base biological quantities outputs specify respective measurements of transcription initiation of the respective target bases at respective positions in the target base sequence.
2. The artificial intelligence-based system of clause 1, wherein the respective measurements of evolutionary conservation are phylogenetic P-values (phyloP) scores that specify a deviation from a null model of neural substitution to detect a reduction in a rate of substitution of a given target base at a given position in the target base sequence as conservation, and to detect an increase in the rate of substitution of the given target base at the given position as acceleration.
3. The artificial intelligence-based system of clause 2, wherein the respective measurements of evolutionary conservation are phastCons scores that specify a posterior probability of the given target base at the given position having a conserved state or a non-conserved state.
4. The artificial intelligence-based system of clause 2, wherein the respective measurements of evolutionary conservation are genomic evolutionary rate profiling (GERP) scores that specify a reduction in a number of substitutions of the given target base at the given position across the plurality of species.
5. The artificial intelligence-based system of clause 1, wherein the respective measurements of transcription initiation are cap analysis of gene expression (CAGE) scores that specify a transcription initiation frequency of the given target base at the given position.
6. The artificial intelligence-based system of clause 1, wherein a third biological quantities output sequence in the plurality of biological quantities output sequences includes a third respective per-base biological quantities outputs for the respective target bases in the target base sequence,
   wherein the third respective per-base biological quantities outputs specify respective measurements of epigenetic signal levels of the respective target bases at respective positions in the target base sequence.
7. The artificial intelligence-based system of clause 6, wherein the epigenetic signal levels specify DNase I-hypersensitive sites (DHSs) or assay for transposase-accessible chromatin with sequencing (ATAC-Seq).
8. The artificial intelligence-based system of clause 6, wherein the epigenetic signal levels specify transcription factor (TF) bindings.
9. The artificial intelligence-based system of clause 6, wherein the epigenetic signal levels specify histone modification (HM) marks.
10. The artificial intelligence-based system of clause 1, further configured to comprise:
   a gene expression model that processes the plurality of biological quantities output sequences and generates an alternative representation of the plurality of biological quantities output sequences; and
   a gene expression output generation logic that processes the alternative representation of the plurality of biological quantities output sequences and generates a gene expression output sequence of respective per-base gene expression outputs for the respective target bases in the target base sequence,
   wherein a given per-base gene expression output in the gene expression output sequence for the given target base at the given position specifies a measure of gene expression level of the given target base at the given position.
11. The artificial intelligence-based system of clause 10, wherein the gene expression level is measured in a per-base metric such as CAGE transcription start site (CTSS).
12. The artificial intelligence-based system of clause 10, wherein the gene expression level is measured in a per-gene metric such as transcripts per million (TPM) or reads per kilobase of transcript (RPKM).
13. The artificial intelligence-based system of clause 10, wherein the gene expression level is measured in a per-gene metric such as fragments per kilobase million (FPKM).
14. The artificial intelligence-based system of clause 1, further configured to comprise a variant classification logic.
15. The artificial intelligence-based system of clause 14, wherein the variant classification logic is further configured to comprise a reference input generation logic that accesses the sequence database and generates a reference base sequence, wherein the reference base sequence includes a reference target base sequence, wherein the reference target base sequence includes a reference base at a position-under-analysis, and wherein the reference base is flanked by a right base sequence with downstream context bases, and a left base sequence with upstream context bases.
16. The artificial intelligence-based system of clause 15, wherein the variant classification logic is further configured to comprise an alternate input generation logic that accesses the sequence database and generates an alternate base sequence, wherein the alternate base sequence includes an alternate target base sequence, wherein the alternate target base sequence includes an alternate base at the position-under-analysis, and wherein the alternate base is flanked by the right base sequence with the downstream context bases, and the left base sequence with the upstream context bases.
17. The artificial intelligence-based system of clause 15, wherein the variant classification logic is further configured to comprise a reference processing logic that causes the biological quantities model to process the reference base sequence and generate an alternative representation of the reference base sequence, and further causes the biological quantities output generation logic to process the alternative representation of the reference base sequence and generate a plurality of reference biological quantities output sequences,
   wherein each reference biological quantities output sequence in the plurality of reference biological quantities output sequences includes respective per-base reference biological quantities outputs for respective reference target bases in the reference target base sequence.
18. The artificial intelligence-based system of clause 17, wherein a first reference biological quantities output sequence in the plurality of reference biological quantities output sequences includes a first respective per-base reference biological quantities outputs for the respective reference target bases in the reference target base sequence,
   wherein the first respective per-base reference biological quantities outputs specify respective measurements of evolutionary conservation of the respective reference target bases across the plurality of species.
19. The artificial intelligence-based system of clause 17, wherein a second reference biological quantities output sequence in the plurality of reference biological quantities output sequences includes a second respective per-base reference biological quantities outputs for the respective reference target bases in the reference target base sequence,
   wherein the second respective per-base reference biological quantities outputs specify respective measurements of transcription initiation of the respective reference target bases at respective positions in the reference target base sequence.
20. The artificial intelligence-based system of clause 16, wherein the variant classification logic is further configured to comprise an alternate processing logic that causes the biological quantities model to process the alternate base sequence and generate an alternative representation of the alternate base sequence, and further causes the biological quantities output generation logic to process the alternative representation of the alternate base sequence and generate a plurality of alternate biological quantities output sequences,
   wherein each alternate biological quantities output sequence in the plurality of alternate biological quantities output sequences includes respective per-base alternate biological quantities outputs for respective alternate target bases in the alternate target base sequence.
21. The artificial intelligence-based system of clause 20, wherein a first alternate biological quantities output sequence in the plurality of alternate biological quantities output sequences includes a first respective per-base alternate biological quantities outputs for the respective alternate target bases in the alternate target base sequence,
   wherein the first respective per-base alternate biological quantities outputs specify respective measurements of evolutionary conservation of the respective alternate target bases across the plurality of species.
22. The artificial intelligence-based system of clause 20, wherein a second alternate biological quantities output sequence in the plurality of alternate biological quantities output sequences includes a second respective per-base alternate biological quantities outputs for the respective alternate target bases in the alternate target base sequence,
   wherein the second respective per-base alternate biological quantities outputs specify respective measurements of transcription initiation of the respective alternate target bases at respective positions in the alternate target base sequence.
23. The artificial intelligence-based system of clause 20, wherein the variant classification logic is further configured to comprise a pathogenicity prediction logic that position-wise compares the first reference biological quantities output sequence and the first alternate biological quantities output sequence and generates a first delta sequence with first position-wise sequence diffs for positions in the first reference biological quantities output sequence and the first alternate biological quantities output sequence.
24. The artificial intelligence-based system of clause 23, wherein the pathogenicity prediction logic is further configured to position-wise compare the second reference biological quantities output sequence and the second alternate biological quantities output sequence and generate a second delta sequence with second position-wise sequence diffs for positions in the second reference biological quantities output sequence and the second alternate biological quantities output sequence.
25. The artificial intelligence-based system of clause 24, wherein the pathogenicity prediction logic is further configured to generate a pathogenicity prediction for the alternate base in dependence upon the first delta sequence and the second delta sequence.
26. The artificial intelligence-based system of clause 24, wherein the pathogenicity prediction logic is further configured to accumulate the first position-wise sequence diffs into a first accumulated sequence value, and to accumulate the second position-wise sequence diffs into a second accumulated sequence value.
27. The artificial intelligence-based system of clause 26, wherein the first accumulated sequence value is an average of the first position-wise sequence diffs, and the second accumulated sequence value is an average of the second position-wise sequence diffs.
28. The artificial intelligence-based system of clause 26, wherein the first accumulated sequence value is a sum of the first position-wise sequence diffs, and the second accumulated sequence value is a sum of the second position-wise sequence diffs.
29. The artificial intelligence-based system of clause 26, wherein the pathogenicity prediction logic is further configured to generate the pathogenicity prediction for the alternate base in dependence upon the first accumulated sequence value and the second accumulated sequence value.
30. The artificial intelligence-based system of clause 29, wherein the pathogenicity prediction logic is further configured to generate the pathogenicity prediction for the alternate base in dependence upon an average of the first accumulated sequence value and the second accumulated sequence value.
31. The artificial intelligence-based system of clause 29, wherein the pathogenicity prediction logic is further configured to generate the pathogenicity prediction for the alternate base in dependence upon a sum of the first accumulated sequence value and the second accumulated sequence value.
32. The artificial intelligence-based system of clause 23, wherein the pathogenicity prediction logic is further configured to classify positions in the first delta sequence as belonging to a conserved state or a non-conserved state based on the first position-wise sequence diffs.
33. The artificial intelligence-based system of clause 32, wherein the pathogenicity prediction logic is further configured to classify those positions in the second delta sequence as belonging to a signal state that coincide with those positions in the first delta sequence that are classified as belonging to the conserved state, and classify those positions in the second delta sequence as belonging to a noise state that coincide with those positions in the first delta sequence that are classified as belonging to the non-conserved state.
34. The artificial intelligence-based system of clause 33, wherein the pathogenicity prediction logic is further configured to accumulate a subset of the second position-wise sequence diffs into a modulated accumulated sequence value,
   wherein second position-wise sequence diffs in the subset of the second position-wise sequence diffs are located at those positions in the second delta sequence that are classified as belonging to the signal state.
35. The artificial intelligence-based system of clause 34, wherein the pathogenicity prediction logic is further configured to generate the pathogenicity prediction for the alternate base in dependence upon the modulated accumulated sequence value.
36. The artificial intelligence-based system of clause 34, wherein the modulated accumulated sequence value is an average of the second position-wise sequence diffs in the subset of the second position-wise sequence diffs.
37. The artificial intelligence-based system of clause 34, wherein the modulated accumulated sequence value is a sum of the second position-wise sequence diffs in the subset of the second position-wise sequence diffs.
38. The artificial intelligence-based system of clause 24, wherein the pathogenicity prediction logic is further configured to position-wise compare respective portions of the first reference biological quantities output sequence and the first alternate biological quantities output sequence and generate a first delta sub-sequence with first position-wise sub-sequence diffs for positions in the respective portions.
39. The artificial intelligence-based system of clause 38, wherein the pathogenicity prediction logic is further configured to position-wise compare respective portions of the second reference biological quantities output sequence and the second alternate biological quantities output sequence and generate a second delta sub-sequence with second position-wise sub-sequence diffs for positions in the respective portions.
40. The artificial intelligence-based system of clause 39, wherein the respective portions span right and left flanking positions around the position-under-analysis.
41. The artificial intelligence-based system of clause 40, wherein the pathogenicity prediction logic is further configured to generate a pathogenicity prediction for the alternate base in dependence upon the first delta sub-sequence and the second delta sub-sequence.
42. The artificial intelligence-based system of clause 40, wherein the pathogenicity prediction logic is further configured to accumulate the first position-wise sub-sequence diffs into a first accumulated sub-sequence value, and to accumulate the second position-wise sub-sequence diffs into a second accumulated sub-sequence value.
43. The artificial intelligence-based system of clause 42, wherein the first accumulated sub-sequence value is an average of the first position-wise sub-sequence diffs, and the second accumulated sub-sequence value is an average of the second position-wise sub-sequence diffs.
44. The artificial intelligence-based system of clause 42, wherein the first accumulated sub-sequence value is a sum of the first position-wise sub-sequence diffs, and the second accumulated sub-sequence value is a sum of the second position-wise sub-sequence diffs.
45. The artificial intelligence-based system of clause 42, wherein the pathogenicity prediction logic is further configured to generate the pathogenicity prediction for the alternate base in dependence upon the first accumulated sub-sequence value and the second accumulated sub-sequence value.
46. The artificial intelligence-based system of clause 45, wherein the pathogenicity prediction logic is further configured to generate the pathogenicity prediction for the alternate base in dependence upon an average of the first accumulated sub-sequence value and the second accumulated sub-sequence value.
47. The artificial intelligence-based system of clause 45, wherein the pathogenicity prediction logic is further configured to generate the pathogenicity prediction for the alternate base in dependence upon a sum of the first accumulated sub-sequence value and the second accumulated sub-sequence value.
48. The artificial intelligence-based system of clause 38, wherein the pathogenicity prediction logic is further configured to classify positions in the first delta sub-sequence as belonging to a conserved state or a non-conserved state based on the first position-wise sub-sequence diffs.
49. The artificial intelligence-based system of clause 48, wherein the pathogenicity prediction logic is further configured to classify those positions in the second delta sub-sequence as belonging to a signal state that coincide with those positions in the first delta sub-sequence that are classified as belonging to the conserved state, and classify those positions in the second delta sub-sequence as belonging to a noise state that coincide with those positions in the first delta sub-sequence that are classified as belonging to the non-conserved state.
50. The artificial intelligence-based system of clause 49, wherein the pathogenicity prediction logic is further configured to accumulate a subset of the second position-wise sub-sequence diffs into a modulated accumulated sub-sequence value,
   wherein second position-wise sub-sequence diffs in the subset of the second position-wise sub-sequence diffs are located at those positions in the second delta sub-sequence that are classified as belonging to the signal state.
51. The artificial intelligence-based system of clause 50, wherein the pathogenicity prediction logic is further configured to generate the pathogenicity prediction for the alternate base in dependence upon the modulated accumulated sub-sequence value.
52. The artificial intelligence-based system of clause 50, wherein the modulated accumulated sub-sequence value is an average of the second position-wise sub-sequence diffs in the subset of the second position-wise sub-sequence diffs.
53. The artificial intelligence-based system of clause 50, wherein the modulated accumulated sub-sequence value is a sum of the second position-wise sub-sequence diffs in the subset of the second position-wise sub-sequence diffs.
54. The artificial intelligence-based system of clause 1, wherein the target base sequence is a coding region of a gene.
55. The artificial intelligence-based system of clause 1, wherein the target base sequence is a non-coding region of a gene.
56. The artificial intelligence-based system of clause 55, wherein the non-coding region spans transcription start sites, five prime untranslated region (UTRs), three prime UTRs, enhancers, and promoters.
56. The artificial intelligence-based system of clause 16, wherein the alternate base is a singleton variant that occurs in only one outlier individual among a cohort of outlier individuals,
   wherein outlier individuals in the cohort of outlier individuals exhibit extremes levels of gene expression.
57. The artificial intelligence-based system of clause 56, wherein the extremes levels of gene expression are determined from tail quantiles of normalized gene expression levels.
58. The artificial intelligence-based system of clause 57, wherein the extremes levels of gene expression include over gene expression and under gene expression.
59. The artificial intelligence-based system of clause 56, wherein the singleton variant is a coding variant.
60. The artificial intelligence-based system of clause 56, wherein the singleton variant is a non-coding variant.
61. The artificial intelligence-based system of clause 60, wherein the non-coding variant is a promoter variant.
62. The artificial intelligence-based system of clause 60, wherein the non-coding variant is an enhancer variant.
62. The artificial intelligence-based system of clause 1, wherein the biological quantities model has a first set of weights,
   wherein the biological quantities output generation logic has a second set of weights.
63. The artificial intelligence-based system of clause 62, wherein, during training, the first set of weights of the biological quantities model is trained from scratch to process the input base sequence and generate the alternative representation of the input base sequence,
   wherein the second set of weights of the biological quantities output generation logic is trained from scratch and end-to-end with the first set of weights of the biological quantities model to process the alternative representation of the input base sequence and generate the plurality of biological quantities output sequences.
64. The artificial intelligence-based system of clause 63, wherein, during inference, the biological quantities model uses the trained first set of weights,
   wherein, during the inference, the biological quantities output generation logic uses the trained second set of weights.
65. The artificial intelligence-based system of clause 1, wherein the gene expression model has a third set of weights,
   wherein the gene expression output generation logic has a fourth set of weights.
66. The artificial intelligence-based system of clause 65, wherein the third set of weights of the gene expression model is trained from scratch to process the plurality of biological quantities output sequences and generate the alternative representation of the plurality of biological quantities output sequences,
   wherein the fourth set of weights of the gene expression output generation logic is trained from scratch and end-to-end with the third set of weights of the gene expression model to process the alternative representation of the plurality of biological quantities output sequences and generate the gene expression output sequence.
67. The artificial intelligence-based system of clause 66, wherein, during inference, the gene expression model uses the trained third set of weights,
   wherein, during the inference, the gene expression output generation logic uses the trained fourth set of weights.
68. The artificial intelligence-based system of clause 65, wherein, during training, the first set of weights of the biological quantities model is first trained from scratch to process the input base sequence and generate the alternative representation of the input base sequence, and then retrained as a substitute of the third set of weights of the gene expression model to process the plurality of biological quantities output sequences and generate the alternative representation of the plurality of biological quantities output sequences,
   wherein the fourth set of weights of the gene expression output generation logic is trained from scratch and end-to-end with the trained first set of weights substituted in the gene expression model to process the alternative representation of the plurality of biological quantities output sequences generated by the trained first set of weights substituted in the gene expression model and generate the gene expression output sequence.
69. The artificial intelligence-based system of clause 68, wherein, during inference, the biological quantities model uses the retrained first set of weights,
   wherein, during the inference, the biological quantities output generation logic uses the trained second set of weights,
   wherein, during inference, the gene expression model uses the retrained first set of weights, and
   wherein, during the inference, the gene expression output generation logic uses the trained fourth set of weights.
70. The artificial intelligence-based system of clause 17, wherein, during training, the first set of weights of the biological quantities model is first trained from scratch to process the input base sequence and generate the alternative representation of the input base sequence,
   wherein, during the training, the second set of weights of the biological quantities output generation logic is first trained from scratch and end-to-end with the first set of weights of the biological quantities model to process the alternative representation of the input base sequence and generate the plurality of biological quantities output sequences,
   wherein, during the training, the trained first set of weights of the biological quantities model is then retrained to process the reference base sequence and generate the alternative representation of the reference base sequence, and to process the alternate base sequence and generate the alternative representation of the alternate base sequence, and
   wherein, during the training, the trained second set of weights of the biological quantities output generation logic is then retrained end-to-end with the trained first set of weights of the biological quantities model to process the alternative representation of the reference base sequence and generate the plurality of reference biological quantities output sequences, and to process the alternative representation of the alternate base sequence and generate the plurality of alternate biological quantities output sequences.
71. The artificial intelligence-based system of clause 70, wherein, during inference, the biological quantities model uses the retrained first set of weights,
   wherein, during the inference, the biological quantities output generation logic uses the retrained second set of weights.
72. The artificial intelligence-based system of clause 23, wherein the pathogenicity prediction logic has a fifth set of weights.
73. The artificial intelligence-based system of clause 72, wherein, during training, the first set of weights of the biological quantities model is first trained from scratch to process the input base sequence and generate the alternative representation of the input base sequence,
   wherein, during the training, the second set of weights of the biological quantities output generation logic is first trained from scratch and end-to-end with the first set of weights of the biological quantities model to process the alternative representation of the input base sequence and generate the plurality of biological quantities output sequences, and
   wherein, during the training, the trained first set of weights of the biological quantities model and the trained second set of weights of the biological quantities output generation logic are then retrained end-to-end to generate the pathogenicity prediction for the alternate base.
74. The artificial intelligence-based system of clause 73, wherein, during inference, the biological quantities model uses the retrained first set of weights,
   wherein, during the inference, the biological quantities output generation logic uses the retrained second set of weights, and
   wherein, during the inference, the pathogenicity prediction logic uses the trained fifth set of weights.
75. The artificial intelligence-based system of clause 18, wherein the first respective per-base reference biological quantities outputs specify respective measurements of first reference epigenetic signal levels of the respective reference target bases at the respective positions in the reference target base sequence.
76. The artificial intelligence-based system of clause 29, wherein the second respective per-base reference biological quantities outputs specify respective measurements of second reference epigenetic signal levels of the respective reference target bases at the respective positions in the reference target base sequence.
77. The artificial intelligence-based system of clause 21, wherein the first respective per-base alternate biological quantities outputs specify respective measurements of first alternate epigenetic signal levels of the respective alternate target bases at the respective positions in the alternate target base sequence.
78. The artificial intelligence-based system of clause 22, wherein the second respective per-base alternate biological quantities outputs specify respective measurements of second alternate epigenetic signal levels of the respective alternate target bases at the respective positions in the alternate target base sequence.
79. The artificial intelligence-based system of clause 1, wherein, during training, the biological quantities model and the biological quantities output generation logic are first trained from scratch and end-to-end to translate analysis of input base sequences into base-wise evolutionary conservation chromatin sequences, and then retrained end-to-end to translate analysis of input base sequences into base-wise transcription initiation frequency chromatin sequences.
80. The artificial intelligence-based system of clause 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise evolutionary conservation chromatin sequences.
81. The artificial intelligence-based system of clause 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise transcription initiation frequency chromatin sequences.
82. The artificial intelligence-based system of clause 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise evolutionary conservation chromatin sequences and base-wise transcription initiation frequency chromatin sequences.
83. The artificial intelligence-based system of clause 1, further configured to comprise a first training set of training input base sequences that include variants confounded by a plurality of epigenetic effects.
84. The artificial intelligence-based system of clause 83, wherein epigenetic effects in the plurality of epigenetic effects include inter-chromosomal effects, intra-gene effects, population structure and ancestry effects, probabilistic estimation of expression residuals (PEER) effects, environmental effects, gender effects, batch effects, genotyping platform effects, and/or library construction protocol effects.
85. The artificial intelligence-based system of clause 83, further configured to comprise a second training set of training input base sequences that include variants unconfounded by the plurality of epigenetic effects.
86. The artificial intelligence-based system of clause 85, wherein the variants in the second training set are reliably determined to alter gene expression and cause extreme levels of gene expression.
87. The artificial intelligence-based system of clause 86, wherein the variants in the second training set include over expression-causing variants that increase gene expression levels.
88. The artificial intelligence-based system of clause 86, wherein the variants in the second training set include under expression-causing variants that decrease gene expression levels.
89. The artificial intelligence-based system of clause 87, wherein the second training set specifies over expression probabilities for the variants that specify likelihoods of the causing gene over expression.
90. The artificial intelligence-based system of clause 88, wherein the second training set specifies under expression probabilities for the variants that specify likelihoods of the variants causing gene under expression.
91. The artificial intelligence-based system of clause 86, wherein each variant in the second training set is a singleton variant that occurs in only one outlier individual among a cohort of outlier individuals, wherein outlier individuals in the cohort of outlier individuals exhibit extremes levels of gene expression.
92. The artificial intelligence-based system of clause 91, wherein the extremes levels of gene expression are determined from tail quantiles of normalized gene expression levels.
93. The artificial intelligence-based system of clause 91, wherein the extremes levels of gene expression include over gene expression and under gene expression.
94. The artificial intelligence-based system of clause 91, wherein the singleton variant is a coding variant.
95. The artificial intelligence-based system of clause 91, wherein the singleton variant is a non-coding variant.
96. The artificial intelligence-based system of clause 95, wherein the non-coding variant is a five prime untranslated region (UTR) variant, a three prime UTR variant, an enhancer variant, or a promoter variant.
97. The artificial intelligence-based system of clause 85, wherein the variants in the second training set span a plurality of tissue types.
98. The artificial intelligence-based system of clause 85, wherein the variants in the second training set span a plurality of cell types.
99. The artificial intelligence-based system of clause 1, wherein the input base sequences and the plurality of biological quantities output sequences span the plurality of tissue types.
100. The artificial intelligence-based system of clause 1, wherein the input base sequences and the plurality of biological quantities output sequences span the plurality of cell types.
101. The artificial intelligence-based system of clause 10, wherein the gene expression output sequence spans the plurality of tissue types.
102. The artificial intelligence-based system of clause 10, wherein the gene expression output sequence spans the plurality of cell types.
103. The artificial intelligence-based system of clause 1, wherein the biological quantities model and the biological quantities output generation logic are first trained end-to-end on the first training set, and then retrained on the second training set.
104. The artificial intelligence-based system of clause 1, wherein the variants in the second training set are used as a pathogenic set labelled with a first ground truth label indicating gene expression alteration, and common variants are used as a benign set labelled with a second ground truth label indicating gene expression non-alteration.
105. The artificial intelligence-based system of clause 104, wherein the benign set is balanced for trinucleotide context, homopolymers, k-mers, neighborhood GC frequency, and sequencing depth.
106. The artificial intelligence-based system of clause 104, wherein, based on a cutoff probability applied to the over expression probabilities and the under expression probabilities, the variants in the second training set are partitioned into an over expression variant training set with a first ground truth label indicating gene expression increase, an over expression variant training set with a second ground truth label indicating gene expression reduction, and a neural expression variant training set indicating gene expression maintenance.
107. The artificial intelligence-based system of clause 10, wherein the gene expression model and the gene expression output generation logic are first trained end-to-end on the first training set, and then retrained on the second training set.
108. The artificial intelligence-based system of clause 1, wherein the biological quantities model and the biological quantities output generation logic are first trained end-to-end on the first training set, and then retrained on those variants in the second training set that occur on odd chromosomes.
109. The artificial intelligence-based system of clause 10, wherein the gene expression model and the gene expression output generation logic are first trained end-to-end on the first training set, and then retrained on those variants in the second training set that occur on odd chromosomes.
110. The artificial intelligence-based system of clause 85, wherein the variants in the second training set are not used for training instead used as a validation set to evaluate performance of the trained biological quantities model 124, the trained the biological quantities output generation logic, the trained gene expression model, and the trained gene expression output generation logic.
111. The artificial intelligence-based system of clause 110, wherein those variants in the second training set that occur on even chromosomes are used as the validation set.
112. The artificial intelligence-based system of clause 1, wherein a size of the target base sequence varies during training to account for varying offset locations of transcription start sites (TSSs).
113. An artificial intelligence-based system to detect changes in gene expression at base resolution, comprising:
   an input generation logic that accesses a sequence database and generates an input base sequence, wherein the input base sequence includes a target base sequence, and wherein the target base sequence is flanked by a right base sequence with downstream context bases, and a left base sequence with upstream context bases;
   a biological quantities model that processes the input base sequence and generates an alternative representation of the input base sequence; and
   a biological quantities output generation logic that processes the alternative representation of the input base sequence and generates a plurality of biological quantities output sequences,
      wherein each biological quantities output sequence in the plurality of biological quantities output sequences includes respective per-base biological quantities outputs for respective target bases in the target base sequence.
114. The artificial intelligence-based system of clause 113, wherein a first biological quantities output sequence in the plurality of biological quantities output sequences includes a first respective per-base biological quantities outputs for the respective target bases in the target base sequence, and
   wherein the first respective per-base biological quantities outputs specify respective measurements of evolutionary conservation of the respective target bases across a plurality of species.
115. The artificial intelligence-based system of clause 113, wherein a second biological quantities output sequence in the plurality of biological quantities output sequences includes a second respective per-base biological quantities outputs for the respective target bases in the target base sequence, and
   wherein the second respective per-base biological quantities outputs specify respective measurements of transcription initiation of the respective target bases at respective positions in the target base sequence.

### Clauses Set 2

1. A system, comprising:
   validation data having a set of variants with a set of causality scores, wherein a causality score specifies a statistically unconfounded likelihood of altering gene expression;
   validation set discretization logic configured to classify each causality score in the set of causality scores to a gene expression altering class or a gene expression preserving class based on an application of a cutoff to the set of causality scores, thereby generating a ground truth bifurcation of the set of variants into the gene expression altering class and the gene expression preserving class;
   inference logic configured to cause a model to generate a set of prediction scores for the set of variants, wherein a prediction score in the set of prediction scores specifies an inferred likelihood of altering gene expression, and wherein the model is trained to determine gene expression alterability of variants;
   model score discretization logic configured to classify each prediction score in the set of prediction scores to the gene expression altering class or the gene expression preserving class based on an application of a threshold to the set of prediction scores, thereby generating an inferred bifurcation of the set of variants into the gene expression altering class and the gene expression preserving class; and
   validation logic configured to determine a performance measure of the model based on a comparison of the inferred bifurcation against the ground truth bifurcation.
2. The system of clause 1, wherein the ground truth bifurcation assigns those variants that are classified to the gene expression altering class a first label (e.g., 0), and assigns those variants that are classified to the gene expression preserving class a second label (e.g., 1).
3. The system of clause 2, wherein the ground truth bifurcation assigns those variants that are classified to the gene expression altering class a first label (e.g., 0), and assigns those variants that are classified to the gene expression preserving class a second label (e.g., 1).
In other implementations, the ground truth bifurcation bifurcates the variants into three categories: −1, 0, 1, corresponding to reducing gene expression, no change in gene expression, and increasing gene expression. That is, a single classifier can do a 3-way classification in such implementations.
4. The system of clause 3, further configured to encode the ground truth bifurcation in a first vector, and to encode the inferred bifurcation in a second vector.
5. The system of clause 4, further configured to determine the performance measure of the model based on an element-wise comparison of the first vector and the second vector.
6. The system of clause 1, further configured to determine the performance measure of the model based on an odds ratio of a number of variants classified by the inferred bifurcation to the gene expression altering class, a number of variants classified by the inferred bifurcation to the gene expression preserving class, a number of variants classified by the ground truth bifurcation to the gene expression altering class, and a number of variants classified by the ground truth bifurcation to the gene expression preserving class.
7. The system of clause 1, wherein the set of variants has a set of under expression causality scores, wherein an under expression causality score specifies a statistically unconfounded likelihood of reducing gene expression.
8. The system of clause 7, wherein the validation set discretization logic is further configured to classify each under expression causality score in the set of under expression causality scores to a gene expression reducing class or a gene expression not reducing class based on an application of an under expression cutoff to the set of under expression causality scores, thereby generating an under expression ground truth bifurcation of the set of variants into the gene expression reducing class and the gene expression not reducing class.
9. The system of clause 8, wherein the inference logic is further configured to cause the model to generate a set of under expression prediction scores for the set of variants, wherein an under expression prediction score in the set of under expression prediction scores specifies an inferred likelihood of reducing gene expression, and wherein the model is trained to determine gene expression reducability of variants,
   wherein the model score discretization logic is further configured to classify each under expression prediction score in the set of under expression prediction scores to the gene expression reducing class or the gene expression not reducing class based on an application of an under expression threshold to the set of under expression prediction scores, thereby generating an under expression inferred bifurcation of the set of variants into the gene expression reducing class and the gene expression not reducing class, and
   wherein the validation logic is further configured to determine an under expression performance measure of the model based on a comparison of the under expression inferred bifurcation against the under expression ground truth bifurcation.
10. The system of clause 9, wherein the model score discretization logic is further configured to sort the set of under expression prediction scores in a decreasing order, to classify a subset of N lowest under expression prediction scores in the sorted set of under expression prediction scores to the gene expression reducing class, and to classify a subset of remaining under expression prediction scores in the sorted set of under expression prediction scores to the gene expression not reducing class.
11. The system of clause 8, wherein the under expression ground truth bifurcation assigns those variants that are classified to the gene expression reducing class a first label (e.g., 0), and assigns those variants that are classified to the gene expression not reducing class a second label (e.g., 1).
12. The system of clause 11, wherein the under expression inferred bifurcation assigns those variants that are classified to the gene expression reducing class the first label (e.g., 0), and assigns those variants that are classified to the gene expression not reducing class the second label (e.g., 1).
13. The system of clause 12, further configured to encode the under expression ground truth bifurcation in a first vector, and to encode the inferred bifurcation in a second vector.
14. The system of clause 13, further configured to determine the under expression performance measure of the model based on an element-wise comparison of the first vector and the second vector.
15. The system of clause 9, further configured to determine the under expression performance measure of the model based on an odds ratio of a number of variants classified by the under expression inferred bifurcation to the gene expression reducing class, a number of variants classified by the under expression inferred bifurcation to the gene expression not reducing class, a number of variants classified by the under expression ground truth bifurcation to the gene expression reducing class, and a number of variants classified by the under expression ground truth bifurcation to the gene expression not reducing class.
16. The system of clause 1, wherein the set of variants has a set of over expression causality scores, wherein an over expression causality score specifies a statistically unconfounded likelihood of increasing gene expression.
17. The system of clause 16, wherein the validation set discretization logic is further configured to classify each over expression causality score in the set of over expression causality scores to a gene expression increasing class or a gene expression not increasing class based on an application of an over expression cutoff to the set of over expression causality scores, thereby generating an over expression ground truth bifurcation of the set of variants into the gene expression increasing class and the gene expression not increasing class.
18. The system of clause 17, wherein the inference logic is further configured to cause the model to generate a set of over expression prediction scores for the set of variants, wherein an over expression prediction score in the set of over expression prediction scores specifies an inferred likelihood of increasing gene expression, and wherein the model is trained to determine gene expression increasability of variants,
   wherein the model score discretization logic is further configured to classify each over expression prediction score in the set of over expression prediction scores to the gene expression increasing class or the gene expression not increasing class based on an application of an over expression threshold to the set of over expression prediction scores, thereby generating an over expression inferred bifurcation of the set of variants into the gene expression increasing class and the gene expression not increasing class, and
   wherein the validation logic is further configured to determine an over expression performance measure of the model based on a comparison of the over expression inferred bifurcation against the over expression ground truth bifurcation.
19. The system of clause 18, wherein the model score discretization logic is further configured to sort the set of over expression prediction scores in a decreasing order, to classify a subset of N highest over expression prediction scores in the sorted set of over expression prediction scores to the gene expression increasing class, and to classify a subset of remaining over expression prediction scores in the sorted set of over expression prediction scores to the gene expression not increasing class.
20. The system of clause 17, wherein the over expression ground truth bifurcation assigns those variants that are classified to the gene expression increasing class a first label (e.g., 0), and assigns those variants that are classified to the gene expression not increasing class a second label (e.g., 1).
21. The system of clause 20, wherein the over expression inferred bifurcation assigns those variants that are classified to the gene expression increasing class the first label (e.g., 0), and assigns those variants that are classified to the gene expression not increasing class the second label (e.g., 1).
22. The system of clause 21, further configured to encode the over expression ground truth bifurcation in a first vector, and to encode the inferred bifurcation in a second vector.
23. The system of clause 22, further configured to determine the over expression performance measure of the model based on an element-wise comparison of the first vector and the second vector.
24. The system of clause 18, further configured to determine the over expression performance measure of the model based on an odds ratio of a number of variants classified by the over expression inferred bifurcation to the gene expression increasing class, a number of variants classified by the over expression inferred bifurcation to the gene expression not increasing class, a number of variants classified by the over expression ground truth bifurcation to the gene expression increasing class, and a number of variants classified by the over expression ground truth bifurcation to the gene expression not increasing class.
25. The system of clause 1, wherein the inference logic is further configured to cause a first model to generate a first set of prediction scores for the set of variants, wherein a prediction score in the first set of prediction scores specifies an inferred likelihood of altering gene expression, and wherein the first model is trained to determine gene expression alterability of variants,
   wherein the model score discretization logic is further configured to classify each prediction score in the first set of prediction scores to the gene expression altering class or the gene expression preserving class based on an application of a first threshold to the first set of prediction scores, thereby generating a first inferred bifurcation of the set of variants into the gene expression altering class and the gene expression preserving class, and
   wherein the validation logic is further configured to determine a first performance measure of the first model based on a comparison of the first inferred bifurcation against the ground truth bifurcation.
26. The system of clause 25, wherein the inference logic is further configured to cause a second model to generate a second set of prediction scores for the set of variants, wherein a prediction score in the second set of prediction scores specifies an inferred likelihood of altering gene expression, and wherein the second model is trained to determine gene expression alterability of variants,
   wherein the model score discretization logic is further configured to classify each prediction score in the second set of prediction scores to the gene expression altering class or the gene expression preserving class based on an application of a second threshold to the second set of prediction scores, thereby generating a second inferred bifurcation of the set of variants into the gene expression altering class and the gene expression preserving class, and
   wherein the validation logic is further configured to determine a second performance measure of the second model based on a comparison of the second inferred bifurcation against the ground truth bifurcation.
27. The system of clause 26, wherein the inference logic is further configured to cause a third model to generate a third set of prediction scores for the set of variants, wherein a prediction score in the third set of prediction scores specifies an inferred likelihood of altering gene expression, and wherein the third model is trained to determine gene expression alterability of variants,
   wherein the model score discretization logic is further configured to classify each prediction score in the third set of prediction scores to the gene expression altering class or the gene expression preserving class based on an application of a third threshold to the third set of prediction scores, thereby generating a third inferred bifurcation of the set of variants into the gene expression altering class and the gene expression preserving class, and
   wherein the validation logic is further configured to determine a third performance measure of the third model based on a comparison of the third inferred bifurcation against the ground truth bifurcation.
28. The system of clause 27, further configured to require the first, second, and third inferred bifurcations to classify a same number of variants in the gene expression altering class, thereby making the first, second, and third performance measures comparable to each other.
29. The system of clause 28, further configured to compare respective performances of the first, second, and third models on the validation data based on a comparison of the first, second, and third performance measures.
30. The system of clause 27, wherein the first, second, and third thresholds are different.
31. The system of clause 27, wherein at least some of the first, second, and third thresholds are same.
32. The system of clause 1, further configured to generate respective ground truth bifurcations of the set of variants into the gene expression altering class and the gene expression preserving class based on respective applications of different cutoffs to the set of causality scores.
33. The system of clause 1, further configured to generate a ground truth trifurcation of the set of variants into a gene expression reducing class, a gene expression increasing class, and a gene expression preserving class.
34. The system of clause 33, further configured to generate an inferred trifurcation of the set of variants into the gene expression reducing class, the gene expression increasing class, and the gene expression preserving class.
35. The system of clause 1, wherein each variant in the set of variants is a singleton variant that occurs in only one outlier individual among a cohort of outlier individuals,
   wherein outlier individuals in the cohort of outlier individuals exhibit extremes levels of gene expression.
36. The system of clause 35, wherein the extremes levels of gene expression are determined from tail quantiles of normalized gene expression levels.
37. The system of clause 35, wherein the extremes levels of gene expression include over gene expression and under gene expression.
38. The system of clause 35, wherein the singleton variant is a coding variant.
39. The system of clause 35, wherein the singleton variant is a non-coding variant.
40. The system of clause 39, wherein the non-coding variant is a five prime untranslated region (UTR) variant, a three prime UTR variant, an enhancer variant, or a promoter variant.
41. The system of clause 1, wherein the set of variants span a plurality of tissue types.
42. The system of clause 1, wherein the set of variants span a plurality of cell types.
43. A system, comprising:
   validation data having a set of observations with a set of ground truth scores;
   validation set discretization logic configured to classify each ground truth score in the set of ground truth scores to a first class or a second class based on an application of a cutoff to the set of ground truth scores, thereby generating a ground truth bifurcation of the set of observations into the first class and the second class;
   inference logic configured to cause a trained model to generate a set of prediction scores for the set of observations;
   model score discretization logic configured to classify each prediction score in the set of prediction scores to the first class or the second class based on an application of a threshold to the set of prediction scores, thereby generating an inferred bifurcation of the set of observations into the first class and the second class; and
   validation logic configured to determine a performance measure of the trained model based on a comparison of the inferred bifurcation against the ground truth bifurcation.
44. The system of clause 43, further configured to generate respective inferred bifurcations for respective models such that each of the respective inferred bifurcations is required to classify a same number of observations to the first class, thereby making respective performance measures of the respective models comparable to each other.
45. The system of clause 44, further configured to compare respective performances of the respective models on the validation data based on a comparison of the respective performance measures.

While the present invention is disclosed by reference to the preferred implementations and examples detailed above, it is to be understood that these examples are intended in an illustrative rather than in a limiting sense. It is contemplated that modifications and combinations will readily occur to those skilled in the art, which modifications and combinations will be within the spirit of the invention and the scope of the following claims.

1. An artificial intelligence-based system to detect changes in gene expression at base resolution, comprising:
an input generation logic that accesses a sequence database and generates an input base sequence, wherein the input base sequence includes a target base sequence, and wherein the target base sequence is flanked by a right base sequence with downstream context bases, and a left base sequence with upstream context bases;
a biological quantities model that processes the input base sequence and generates an alternative representation of the input base sequence; and
a biological quantities output generation logic that processes the alternative representation of the input base sequence and generates a plurality of biological quantities output sequences,
wherein a first biological quantities output sequence in the plurality of biological quantities output sequences includes a first respective per-base biological quantities outputs for the respective target bases in the target base sequence,
wherein the first respective per-base biological quantities outputs specify respective measurements of evolutionary conservation of the respective target bases across a plurality of species, and
wherein a second biological quantities output sequence in the plurality of biological quantities output sequences includes a second respective per-base biological quantities outputs for the respective target bases in the target base sequence,
wherein the second respective per-base biological quantities outputs specify respective measurements of transcription initiation of the respective target bases at respective positions in the target base sequence.

2. The artificial intelligence-based system of claim 1, wherein the respective measurements of evolutionary conservation are phylogenetic P-values (phyloP) scores that specify a deviation from a null model of neural substitution to detect a reduction in a rate of substitution of a given target base at a given position in the target base sequence as conservation, and to detect an increase in the rate of substitution of the given target base at the given position as acceleration.

3-4. (canceled)

5. The artificial intelligence-based system of claim 1, wherein the respective measurements of transcription initiation are cap analysis of gene expression (CAGE) scores that specify a transcription initiation frequency of the given target base at the given position.

6. The artificial intelligence-based system of claim 1, wherein a third biological quantities output sequence in the plurality of biological quantities output sequences includes a third respective per-base biological quantities outputs for the respective target bases in the target base sequence,
wherein the third respective per-base biological quantities outputs specify respective measurements of epigenetic signal levels of the respective target bases at respective positions in the target base sequence.

7-9. (canceled)

10. The artificial intelligence-based system of claim 1, further configured to comprise:
a gene expression model that processes the plurality of biological quantities output sequences and generates an alternative representation of the plurality of biological quantities output sequences; and
a gene expression output generation logic that processes the alternative representation of the plurality of biological quantities output sequences and generates a gene expression output sequence of respective per-base gene expression outputs for the respective target bases in the target base sequence,
wherein a given per-base gene expression output in the gene expression output sequence for the given target base at the given position specifies a measure of gene expression level of the given target base at the given position.

11-13. (canceled)

14. The artificial intelligence-based system of claim 1, further configured to comprise a variant classification logic.

15-53. (canceled)

54. The artificial intelligence-based system of claim 1, wherein the target base sequence is a coding region of a gene.

55. The artificial intelligence-based system of claim 1, wherein the target base sequence is a non-coding region of a gene.

56-63. (canceled)

64. The artificial intelligence-based system of claim 1, wherein the biological quantities model has a first set of weights,
wherein the biological quantities output generation logic has a second set of weights.

65-66. (canceled)

67. The artificial intelligence-based system of claim 1, wherein the gene expression model has a third set of weights,
wherein the gene expression output generation logic has a fourth set of weights.

68-80. (canceled)

81. The artificial intelligence-based system of claim 1, wherein, during training, the biological quantities model and the biological quantities output generation logic are first trained from scratch and end-to-end to translate analysis of input base sequences into base-wise evolutionary conservation chromatin sequences, and then retrained end-to-end to translate analysis of input base sequences into base-wise transcription initiation frequency chromatin sequences.

82. The artificial intelligence-based system of claim 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise evolutionary conservation chromatin sequences.

83. The artificial intelligence-based system of claim 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise transcription initiation frequency chromatin sequences.

84. The artificial intelligence-based system of claim 1, wherein, during training, the biological quantities model and the biological quantities output generation logic are first trained from scratch and end-to-end to translate analysis of input base sequences into base-wise epigenetic signal level chromatin sequences, and then retrained end-to-end to translate analysis of input base sequences into base-wise evolutionary conservation chromatin sequences and base-wise transcription initiation frequency chromatin sequences.

85. The artificial intelligence-based system of claim 1, further configured to comprise a first training set of training input base sequences that include variants confounded by a plurality of epigenetic effects.

86-100. (canceled)

101. The artificial intelligence-based system of claim 1, wherein the input base sequences and the plurality of biological quantities output sequences span the plurality of tissue types.

102. The artificial intelligence-based system of claim 1, wherein the input base sequences and the plurality of biological quantities output sequences span the plurality of cell types.

103-104. (canceled)

105. The artificial intelligence-based system of claim 1, wherein the biological quantities model and the biological quantities output generation logic are first trained end-to-end on the first training set, and then retrained on the second training set.

106-113. (canceled)

114. The artificial intelligence-based system of claim 1, wherein a size of the target base sequence varies during training to account for varying offset locations of transcription start sites (TSSs).

115. An artificial intelligence-based system to detect changes in gene expression at base resolution, comprising:
an input generation logic that accesses a sequence database and generates an input base sequence, wherein the input base sequence includes a target base sequence, and wherein the target base sequence is flanked by a right base sequence with downstream context bases, and a left base sequence with upstream context bases;
a biological quantities model that processes the input base sequence and generates an alternative representation of the input base sequence; and
a biological quantities output generation logic that processes the alternative representation of the input base sequence and generates a plurality of biological quantities output sequences,
wherein each biological quantities output sequence in the plurality of biological quantities output sequences includes respective per-base biological quantities outputs for respective target bases in the target base sequence.

116-117. (canceled)
